(19) United States
(12) Patent Application Publication
(10) Pub. No.: US 2013/0096316 A1
(43) Pub. Date: Apr. 18, 2013

(54) Novel Method for Preparation of Bisnorcymerine and Salts Thereof

(71) Applicant: Maria MacCecchini, West Chester, PA (US)

(72) Inventor: Maria MacCecchini, West Chester, PA (US)

(73) Assignee: QR PHARMA, INC.

(21) Appl. No.: 13/274,065

(22) Filed: Oct. 14, 2011

(51) Int. Cl. C07D 487/04

(52) U.S. Cl. 548/429

(57) ABSTRACT

The present invention includes a novel method of preparing N¹,N⁸-bisnorcymserine. The present invention also includes a method of preparing a 1:1 salt of (−)-N¹,N⁸-bisnorcymserine with an acid.

### CROSS-REFERENCE TO RELATED APPLICATION

The present application claims priority under 35 U.S.C. §119(e) to U.S. Provisional Application No. 61/407,743, filed Oct. 28, 2010, which application is incorporated by reference herein in its entirety.

### BACKGROUND OF THE INVENTION

Alzheimer's disease (AD) is the most common progressive dementia associated with aging. The major neuropathological hallmarks of the disease are β-amyloid plaques, neurofibrillary tangles, and synaptic loss (Khachaturian, 1985, Arch. Neurol, 42:1095-1105). The cholinergic system is the earliest and most profoundly affected neurotransmitter system in AD, with substantial losses in the forebrain, cortex, and hippocampus, which are critical in the acquisition, processing, and storage of memories (Terry et al., 1991, Ann. Neurol. 30:572-80; Giacobini, In “Alzheimer's Disease: Molecular Biology to Therapy”; Becker & Giacobini, Eds.; Birkhauser: Boston, 1997; pp 188-204; Becker et al., In “Alzheimer's Disease: from Molecular Biology to Therapy”; Becker & Giacobini, Eds.; Birkhauser: Boston, 1997; pp 257-66). Currently, the only agents that have demonstrated efficacy in cellular and animal AD preclinical models that have effectively translated to clinical efficacy (albeit primarily symptomatic) in AD patients are anticholinesterases and the NMDA glutamatergic antagonist, memantine.

Two forms of cholinesterase coexist ubiquitously throughout the body, acetylcholinesterase (AChE; EC 3.1.1.7) and butyrylcholinesterase (BChE; EC 3.1.1.8). Although highly homologous, >65%, AChE and BChE are products of different genes on chromosomes 7 and 3 in humans, respectively (Soreq et al., “Human Cholinesterases and Anticholinesterases”; Academic Press: New York, 1993). Both subtype-unselective cholinesterase and AChE selective inhibitors have been used in AD to amplify the action of acetylcholine (ACh) at remaining cholinergic synapses within the AD brain, and this has promoted the synthesis and development of novel inhibitors of AChE with favorable characteristics for in vivo use by the pharmaceutical industry. The role of BChE in normal, aging, and diseased brain remains largely unknown, and there has been minimal interest in the design, synthesis, and development of selective inhibitors of BChE, except in the agricultural industry where toxic irreversible BChE inhibitors have long been used as insecticides (Giacobini, In “Alzheimer's Disease: Molecular Biology to Therapy”; Becker & Giacobini, Eds.; Birkhauser: Boston, 1997; pp 188-204).

Mounting evidence suggests that inappropriate BChE activity increases the risk and/or progression rate of AD (Guillozet et al., Ann. Neurol. 1997, 42:909-18; Barber et al., 1996, Proc. Soc. Neurosci. 22:1172; Lehman et al., 1997, Hum, Mol. Genet. 6:1933-36). Therefore, well-tolerated inhibitors of BChE may have utility in the treatment of AD.

(−)—N¹,N⁸-Bisnorcymserine (1) was reported as one of the most potent and selective inhibitors of human BChE in an initial pharmacological evaluation (U.S. Pat. Nos. 6,410,747 and 6,683,105). Compound 1 was shown to have a 110-fold selectivity for inhibition of BChE as compared to AChE, with IC₅₀ values of 1.0±0.1 nM, and 110±15 nM, respectively (Yu et al., 1999, J. Med. Chem., 42:1855-61).

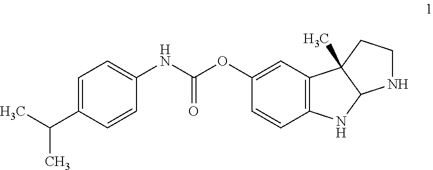 [SMILES: CC(C)c1ccc(NC(=O)Oc2ccc3c(c2)[C@]2(C)CCNC2N3)cc1]

Further characterization of compound (1), including its evaluation in pre-clinical and clinical trials, requires large amounts of drug material, which may only be prepared using a synthetic route that is dependable, scalable and economical. The published synthetic routes for compound (1) are well validated but may not be easily scalable, especially because the last step in the published syntheses is the palladium-catalyzed removal of two benzyl groups from the intermediate (−)-(3aS)-1,8-dibenzyl-3a-methyl-1,2,3,3a,8,8a-hexahydropyrrol[2,3-b]indol-5-yl N-4% isopropylphenyl carbamate (Yu et al., 1999, J. Med. Chem. 42:1855-61). This step generates low yield of product (˜50% of crude product isolated as a gum; see U.S. Pat. Nos. 6,410,747 and 6,683,105), most likely because of the difficult separation of the polar deprotected product and the palladium catalyst. Furthermore, the use of palladium as a reagent in the last step of the synthesis of 1 is not optimal, since the final product should be as free of metallic impurities as possible to be used in vivo.

There is a need in the art to develop a novel synthetic route for pure (−)-N¹,N⁸-bisnorcymserine 1 that may be utilized to prepare large amounts of this compound, or a salt thereof, for pharmacological and clinical studies and treatment of patients. This synthetic route should be easily scalable, reliable and minimize the risk of contamination of the final product with undesirable impurities. The present invention fulfills this need.

### BRIEF SUMMARY OF THE INVENTION

The invention includes a composition comprising a compound of formula 10:

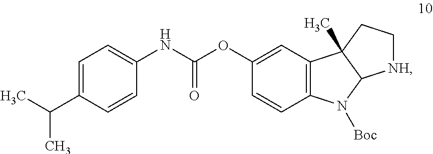 [SMILES: CC(C)c1ccc(NC(=O)Oc2ccc3c(c2)[C@]2(C)CCNC2N3C(=O)OC(C)(C)C)cc1]

or a salt thereof.

The invention also includes a method of preparing a salt comprising an acid and a compound of formula 1:

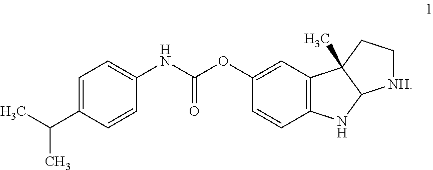 [SMILES: CC(C)c1ccc(NC(=O)Oc2ccc3c(c2)[C@]2(C)CCNC2N3)cc1]

The method comprises the step of dissolving one equivalent of the compound of formula 1 in a first volume of a first solvent, to generate a first solution. The method further comprises the step of dissolving a number of equivalents of the acid in a second volume of a second solvent, to generate a second solution. The method further comprises the step of contacting the second solution with the first solution under stirring, to generate a first system comprising a first solid. The method further comprises the step of stirring the first system at a first temperature for a first period of time. The method further comprises the step of isolating the first solid from the first system by filtration. The method further comprises the step of washing the first solid with a third volume of a third solvent, to generate a second solid. The method further comprises the step of washing the second solid with a fourth volume of a fourth solvent, to generate a third solid. The method further comprises the step of washing the third solid with a fifth volume of a fifth solvent, to generate a fourth solid. The method further comprises the step of isolating and removing volatiles from the fourth solid, to generate the salt.

In one embodiment, the number of equivalents of the acid ranges from about 1 to about 3. In another embodiment, in the salt the ratio of the acid to the compound of formula 1 ranges from about 1:1 to about 3:1. In yet another embodiment, the acid is L-tartaric acid. In yet another embodiment, in the salt the ratio of L-tartaric acid to the compound of formula 1 is about 1:1. In yet another embodiment, the first solvent and the second solvent each comprise isopropanol. In yet another embodiment, the first volume and the second volume are about 5 volumes each. In yet another embodiment, the first temperature ranges from about 45 to about 75° C. and the first period of time is about one hour. In yet another embodiment, the third solvent comprises isopropanol and the third volume is about 3 volumes. In yet another embodiment, the fourth solvent comprises 10 volumes of DMSO and 22 volumes of water. In yet another embodiment, the fifth solvent comprises acetonitrile and the fifth volume is about 11 volumes. In yet another embodiment, the volatiles are removed by spray-drying or freeze-drying the fourth solid.

The invention further includes a method of preparing a compound of formula 1:

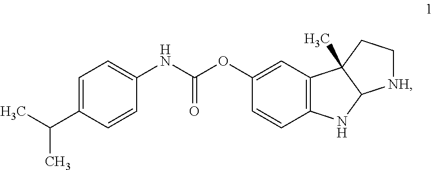 [SMILES: CC(C)c1ccc(NC(=O)Oc2ccc3c(c2)[C@]2(C)CCNC2N3)cc1]

or a salt thereof, comprising the step of hydrolyzing a compound of formula 10:

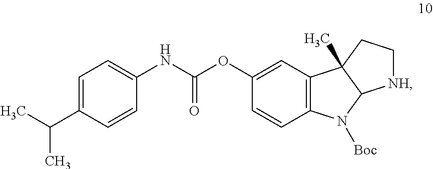 [SMILES: CC(C)c1ccc(NC(=O)Oc2ccc3c(c2)[C@]2(C)CCNC2N3C(=O)OC(C)(C)C)cc1]

or a salt thereof.

In one embodiment, the compound of formula 10 is hydrolyzed with a solution of trifluoroacetic acid in dichloromethane.

In one embodiment, the compound of formula 10 or a salt thereof is prepared from a compound of formula 9 or a salt thereof:

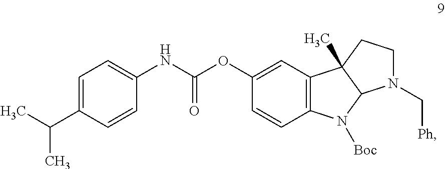 [SMILES: CC(C)c1ccc(NC(=O)Oc2ccc3c(c2)[C@]2(C)CCN(Cc4ccccc4)C2N3C(=O)OC(C)(C)C)cc1]

by hydrogenating the compound of formula 9.

In one embodiment, the compound of formula 9 or a salt thereof is prepared from a compound of formula 8 or a salt thereof:

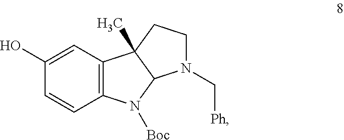 [SMILES: CC(C)(C)OC(=O)N1c2ccc(O)cc2[C@]2(C)CCN(Cc3ccccc3)C12]

by reacting the compound of formula 8 with:
(i) isopropyl isocyanate in the presence of an organic base; or,
(ii) a reagent selected from the group consisting of phosgene, diphosphene, triphosgene, carbonyldiimidazole and para-nitrophenyl chloroformate, to form an intermediate, and further reacting the intermediate with para-isopropylaniline.

In one embodiment, the compound of formula 8 or a salt thereof is prepared from a compound of formula 7 or a salt thereof:

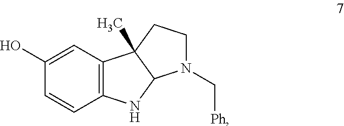 [SMILES: C[C@@]12CCN(Cc3ccccc3)C1Nc1ccc(O)cc12]

by reacting the compound of formula 7 with a BOC-protecting reagent.

In one embodiment, the compound of formula 7 or a salt thereof is prepared from a compound of formula 6 or a salt thereof:

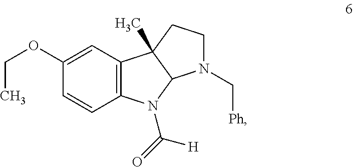 [SMILES: CCOc1ccc2c(c1)[C@]1(C)CCN(Cc3ccccc3)C1N2C=O]

by reacting the compound of formula 6 with a reagent selected from the group consisting of boron tribromide, trimethylsilyl iodide, trimethylsilyl chloride, trifluoroboron etherate, tetrachlorosilane, aluminum tribromide, aluminum trichloride, ferric trichloride, and bromodimethylborane.

In one embodiment, the compound of formula 6 or a salt thereof is prepared from a compound of formula 5 or a salt thereof:

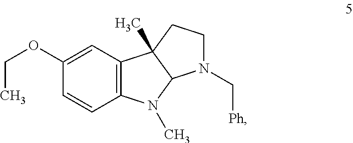 [SMILES: CCOc1ccc2c(c1)[C@]1(C)CCN(Cc3ccccc3)C1N2C]

by reacting the compound of formula 5 with an oxidizer.

In one embodiment, the compound of formula 5 or a salt thereof is prepared from a compound of formula 4 or a salt thereof:

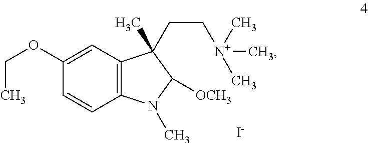 [SMILES: CCOc1ccc2c(c1)[C@](C)(CC[N+](C)(C)C)C(OC)N2C ; [I-]]

by reacting the compound of formula 4 with benzylamine.

In one embodiment, the compound of formula 4 or a salt thereof is prepared from a compound of formula 3 or a salt thereof:

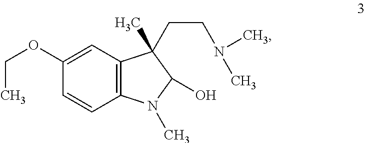 [SMILES: CCOc1ccc2c(c1)[C@](C)(CCN(C)C)C(O)N2C]

by reacting the compound of formula 3 with methyl iodide.

In one embodiment, the compound of formula 3 or a salt thereof is prepared from a compound of formula 2 or a salt thereof:

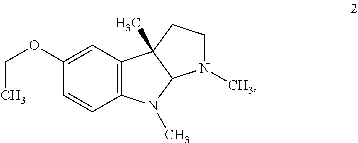 [SMILES: CCOc1ccc2c(c1)[C@]1(C)CCN(C)C1N2C]

comprising the steps of:
(i) reacting the compound of formula 2 with a methylating agent, to generate a methylated derivative of the compound of formula 2, and
(ii) further reacting the methylated derivative with an aqueous solution comprising an inorganic base,
   whereby generating the compound of formula 3.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a drawing illustrating a TLC separation performed for Preparative Example 1.

FIG. 2 is an illustrative representation of a ¹H NMR spectrum obtained for Compound 3.

FIG. 3 is an illustrative representation of a ¹H NMR spectrum obtained for Compound 4.

FIG. 4 is a drawing illustrating a TLC separation performed for Preparative Example 3.

FIG. 5 is an illustrative representation of a ¹H NMR spectrum obtained for Compound 5.

FIG. 6 is an illustrative representation of a HPLC trace obtained for Compound 5.

FIG. 7 is a drawing illustrating a TLC separation performed for Preparative Example 4.

FIG. 8 is an illustrative representation of a ¹H NMR spectrum obtained for Compound 6.

FIG. 9 is an illustrative representation of a LC-MS trace obtained for Compound 6.

FIG. 10 is a drawing illustrating a TLC separation performed for Preparative Example 5.

FIG. 11 is an illustrative representation of a ¹H NMR spectrum obtained for Compound 7.

FIG. 12 is an illustrative representation of a LC-MS trace obtained for Compound 7.

FIG. 13 is a drawing illustrating a TLC separation performed for Preparative Example 6.

FIG. 14 is an illustrative representation of a ¹H NMR spectrum obtained for Compound 8.

FIG. 15 is a drawing illustrating a TLC separation performed for Preparative Example 7.

FIG. 16 is an illustrative representation of a ¹H NMR spectrum obtained for Compound 9.

FIG. 17 is an illustrative representation of a LC-MS trace obtained for Compound 9.

FIG. 18 is a drawing illustrating a TLC separation performed for Preparative Example 8.

FIG. 19 is an illustrative representation of a ¹H NMR spectrum obtained for Compound 10.

FIG. 20 is an illustrative representation of a LC-MS trace obtained for Compound 10.

FIG. 21 is an illustrative representation of a HPLC trace obtained for the reaction in Example 1.

FIG. 22 is a drawing illustrating a TLC separation performed for Example 1.

FIG. 23 is an illustrative representation of a ¹H NMR spectrum obtained for a crude material isolated in Example 1.

FIG. 24 is an illustrative representation of a ¹H NMR spectrum obtained for a purified material isolated in Example 1.

FIG. 25 is an illustrative representation of a ¹H NMR spectrum obtained for Compound 11.

### DETAILED DESCRIPTION OF THE INVENTION

The present invention relates to the discovery of a novel synthetic route that allows the high-yield synthesis of (−)-N¹,N⁸-bisnorcymserine or a salt thereof. This route is easily scalable and reliable, and minimizes the risk of contamination of the final product with undesirable impurities as compared to previously disclosed synthetic routes.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in pharmaceutical chemistry and organic chemistry are those well known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

### COMPOSITIONS OF THE INVENTION

The invention includes a composition comprising a compound of formula 10:

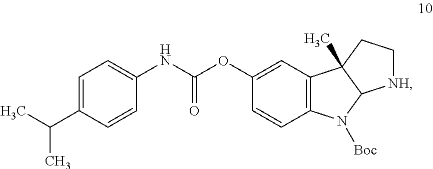 [SMILES: CC(C)c1ccc(NC(=O)Oc2ccc3c(c2)[C@]2(C)CCNC2N3C(=O)OC(C)(C)C)cc1]

or a salt thereof. The compound of formula 10 is useful within the methods of the invention in the preparation of the compound of formula 1, as described below.

The invention further includes a composition comprising a salt,

wherein the salt comprises a compound of formula 1 and an acid, wherein in the salt the ratio of the acid to the compound of formula 1 ranges from about 1:1 to about 3:1. In another embodiment, in the salt the ratio of the acid to the compound of formula 1 ranges from about 1:1 to about 2.5:1. In yet another embodiment, in the salt the ratio of the acid to the compound of formula 1 ranges from about 1:1 to about 2:1. In yet another embodiment, in the salt the ratio of the acid to the compound of formula 1 ranges from about 1:1 to about 1.5:1. In yet another embodiment, in the salt the ratio of the acid to the compound of formula 1 is about 1:1. In yet another embodiment, in the salt the ratio of the acid to the compound of formula 1 is about 1.5:1. In yet another embodiment, in the salt the ratio of the acid to the compound of formula 1 is about 1.6:1. In yet another embodiment, in the salt the ratio of the acid to the compound of formula 1 is about 1, 8:1. In yet another embodiment, in the salt the ratio of the acid to the compound of formula 1 is about 2:1.

### METHODS OF THE INVENTION

The invention includes a method of preparing a salt comprising a acid and a compound of formula 1:

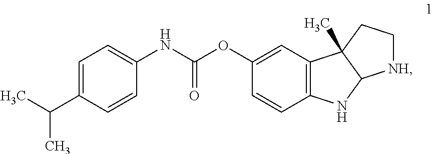 [SMILES: CC(C)c1ccc(NC(=O)Oc2ccc3c(c2)[C@]2(C)CCNC2N3)cc1]

comprising the steps of;
(i) dissolving one equivalent of the compound of formula 1 in a first volume of a first solvent, to generate a first solution;
(ii) dissolving a number of equivalents of the acid in a second volume of a second solvent, to generate a second solution;
(iii) contacting the second solution with the first solution under stirring, to generate a first system comprising a first solid;
(iv) stirring the first system at a first temperature for a first period of time;
(v) isolating the first solid from the first system by filtration;
(vi) washing the first solid with a third volume of a third solvent, to generate a second solid;
(vii) washing the second solid with a fourth volume of a fourth solvent, to generate a third solid;
(viii) washing the third solid with a fifth volume of a firth solvent, to generate a fourth solid; and,
(ix) isolating and removing volatiles from the fourth solid, to generate the salt.

In one embodiment, the acid is L-tartaric acid.

In one embodiment, the number of equivalents of the acid ranges from about 1 to about 3. In another embodiment, the equivalent of the acid ranges from about 1 to about 2. In yet another embodiment, the equivalent of the acid ranges from about 1 to about 1.5.

In one embodiment, in the salt the ratio of the acid to the compound of formula 1 ranges from about 1:1 to about 3:1. In another embodiment, in the salt the ratio of the acid to the compound of formula 1 ranges from about 1:1 to about 2, 5:1. In yet another embodiment, in the salt the ratio of the acid to the compound of formula 1 ranges from about 1:1 to about 2:1. In yet another embodiment, in the salt the ratio of the acid to the compound of formula 1 ranges from about 1:1 to about 1.5:1. In yet another embodiment, in the salt the ratio of the acid to the compound of formula 1 is about 1:1. In yet another embodiment, in the salt the ratio of the acid to the compound of formula 1 is about 1.5:1. In yet another embodiment, in the salt the ratio of the acid to the compound of formula 1 is about 1.6:1. In yet another embodiment, in the salt the ratio of the acid to the compound of formula 1 is about 1, 8:1. In yet another embodiment, in the alt the ratio of the acid to the compound of formula 1 is about 2:1.

In one embodiment, the acid is tartaric acid, and in the salt the ratio of the tartaric acid to the compound of formula 1 is about 1:1:

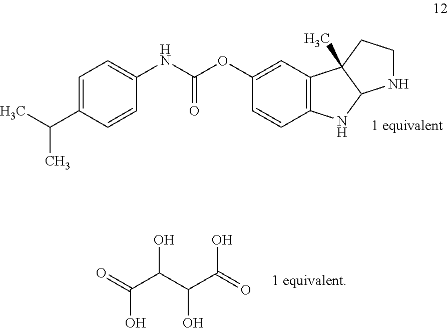 [SMILES: CC(C)c1ccc(NC(=O)Oc2ccc3c(c2)[C@]2(C)CCNC2N3)cc1 ; O=C(O)C(O)C(O)C(=O)O]

In one embodiment, the first solvent and the second solvent each comprise isopropanol. In another embodiment, the first volume and the second volume are about 5 volumes each. In yet another embodiment, the first temperature ranges from about 45 to about 75° C. and the first period of time is about one hour. In yet another embodiment, the third solvent comprises isopropanol and the third volume is about 3 volumes. In yet another embodiment, the fourth solvent comprises 10 volumes of DMSO and 22 volumes of water. In yet another embodiment, the fifth solvent comprises acetonitrile and the fifth volume is about 11 volumes. In yet another embodiment, the volatiles are removed by freeze-drying the fourth solid. In yet another embodiment, the volatiles are removed by spray-drying the fourth solid.

The invention further includes a method of preparing a compound of formula:

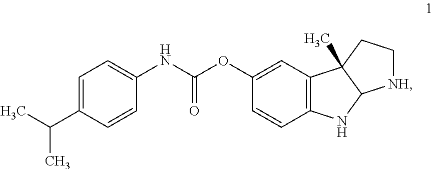 [SMILES: CC(C)c1ccc(NC(=O)Oc2ccc3c(c2)[C@]2(C)CCNC2N3)cc1]

or a salt thereof, comprising the step of hydrolyzing a compound of formula:

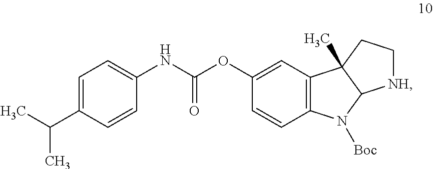 [SMILES: CC(C)c1ccc(NC(=O)Oc2ccc3c(c2)[C@]2(C)CCNC2N3C(=O)OC(C)(C)C)cc1]

or a salt thereof.

In one embodiment, the compound of formula 10 is hydrolyzed with a solution of trifluoroacetic acid in dichloromethane.

In one embodiment, the compound of formula 10 or a salt thereof is prepared from a compound of formula:

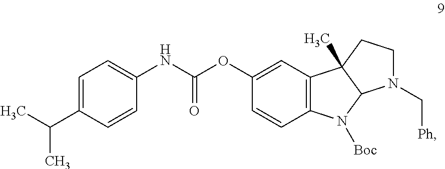 [SMILES: CC(C)c1ccc(NC(=O)Oc2ccc3c(c2)[C@]2(C)CCN(Cc4ccccc4)C2N3C(=O)OC(C)(C)C)cc1]

or a salt thereof, by hydrogenating the compound of formula 9.

In one embodiment, the compound of formula 9 or a salt thereof is prepared from a compound of formula:

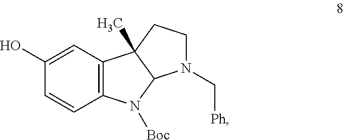 [SMILES: CC(C)(C)OC(=O)N1c2ccc(O)cc2[C@]2(C)CCN(Cc3ccccc3)C12]

or a salt thereof; by reacting the compound of formula 8 with:
(i) isopropyl isocyanate in the presence of an organic base; or,
(ii) a reagent selected from the group consisting of phosgene, diphosphene, triphosgene, carbonyldilmidazole and para-nitrophenyl chloroformate, to form an intermediate, and further reacting the intermediate with para-isopropylaniline.

In one embodiment, the compound of formula 8 or a salt thereof is prepared from a compound of formula:

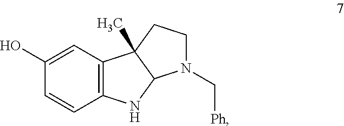 [SMILES: C[C@@]12CCN(Cc3ccccc3)C1Nc1ccc(O)cc12]

or a salt thereof, by reacting the compound of formula 7 with a BOC-protecting reagent.

In one embodiment, the compound of formula 7 or a salt thereof is prepared from a compound of formula:

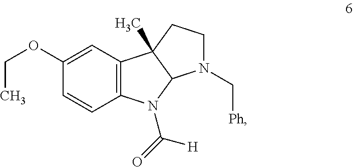 [SMILES: CCOc1ccc2c(c1)[C@]1(C)CCN(Cc3ccccc3)C1N2C=O]

or a salt thereof, by reacting the compound of formula 6 with a reagent selected from the group consisting of boron tribromide, trimethylsilyl iodide, trimethylsilyl chloride, trifluoroboron etherate, tetrachlorosilane, aluminum tribromide, aluminum trichloride, ferric trichloride, and bromodimethylborane.

In one embodiment, the compound of formula 6 or a salt thereof is prepared from a compound of formula:

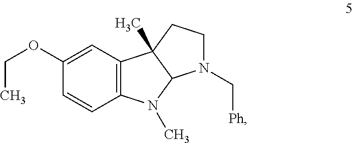 [SMILES: CCOc1ccc2c(c1)[C@]1(C)CCN(Cc3ccccc3)C1N2C]

or a salt thereof, by reacting the compound of formula 5 with an oxidizer.

In one embodiment, the compound of formula 5 or a salt thereof is prepared from a compound of formula:

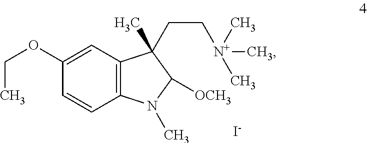 [SMILES: CCOc1ccc2c(c1)[C@](C)(CC[N+](C)(C)C)C(OC)N2C ; [I-]]

or a salt thereof, by reacting the compound of formula 4 with benzylamine.

In one embodiment, the compound of formula 4 or a salt thereof is prepared from a compound of formula:

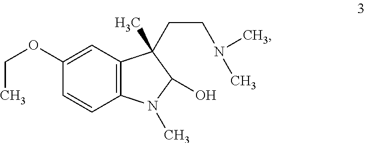 [SMILES: CCOc1ccc2c(c1)[C@](C)(CCN(C)C)C(O)N2C]

or a salt thereof, by reacting the compound of formula 3 with methyl iodide.

In one embodiment, the compound of formula 3 or a salt thereof is prepared from a compound of formula:

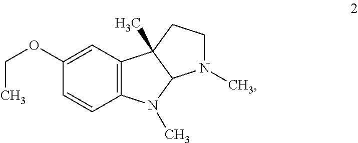 [SMILES: CCOc1ccc2c(c1)[C@]1(C)CCN(C)C1N2C]

or a salt thereof, comprising the steps of:
(i) reacting the compound of formula 2 with a methylating agent, to generate a methylated derivative of the compound of formula 2, and
(ii) further reacting the methylated derivative with an aqueous solution comprising an inorganic base,
   whereby generating the compound of formula 3.

In one aspect, compound 1 and a salt thereof, such as the tartrate salt 11, may be prepared according to the following synthetic scheme:

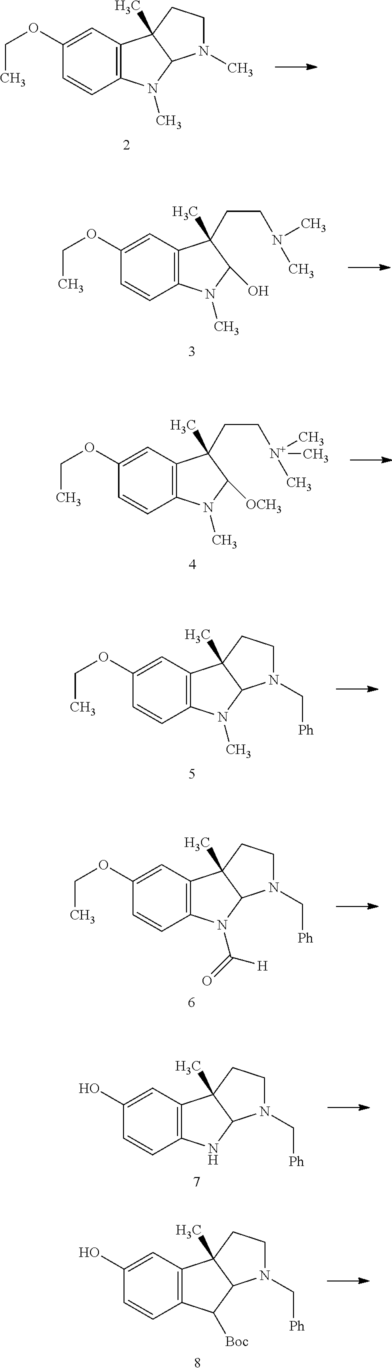

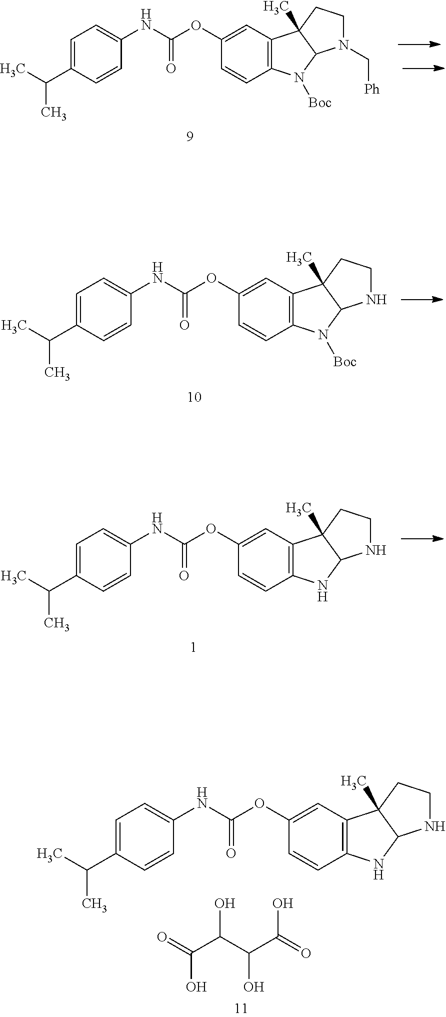 [SMILES: CC(C)c1ccc(NC(=O)Oc2ccc3c(c2)[C@]2(C)CCN(Cc4ccccc4)C2N3C(=O)OC(C)(C)C)cc1 ; CC(C)c1ccc(NC(=O)Oc2ccc3c(c2)[C@]2(C)CCNC2N3C(=O)OC(C)(C)C)cc1 ; CC(C)c1ccc(NC(=O)Oc2ccc3c(c2)[C@]2(C)CCNC2N3)cc1 ; CC(C)c1ccc(NC(=O)Oc2ccc3c(c2)[C@]2(C)CCNC2N3)cc1 ; O=C(O)C(O)C(O)C(=O)O]

In one aspect, compound 2, also known as (3aS)-5-ethoxy-1,3a,8-trimethyl-1,2,3,3a,8,8a-hexahydropyrrolo[2,3-b]indole or (−)-eserethole, is useful within the methods of the invention. Compound 2 may be prepared according to methods described in the literature (see, for example, U.S. Pat. Nos. 5,519,144 and

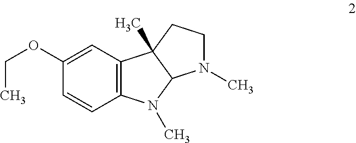 [SMILES: CCOc1ccc2c(c1)[C@]1(C)CCN(C)C1N2C]

Compound 2 may be converted to Compound 3 ((3S)-3-(2-(dimethylamino)ethyl)-5-ethoxy-1,3-dimethylindolin-2-ol) by methylation, followed by basic hydrolysis.

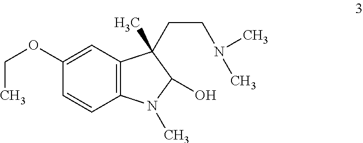 [SMILES: CCOc1ccc2c(c1)[C@](C)(CCN(C)C)C(O)N2C]

The methylation of Compound 2 may be performed with a methylation agent such as, but not limited to, methyl iodide, methyl bromide, methyl triflate or methyl mesylate. The reaction may be performed in an organic solvent that is partially soluble or insoluble in water, such as an ether (as non-limiting examples, diethyl ether or tetrahydrofuran). Progress of the reaction may be followed by a method such as TLC, HPLC or NMR. Once deemed sufficiently complete, the reaction mixture may be concentrated under vacuum and treated with an aqueous solution comprising an inorganic base, such as but not limited to sodium carbonate, sodium bicarbonate, sodium hydroxide or sodium phosphate. Progress of the reaction may be followed by a method such as TLC, HPLC or NMR. The product of the reaction may be isolated by concentration or filtration of the reaction mixture. In a non-limiting embodiment, Compound 3 may be isolated in sufficient purity to be used as is in the next step. In another non-limiting embodiment, Compound 3 may be purified by any purification method known to one skilled in the art, such as but not limited to preparative HPLC, silica gel column chromatography or crystallization from a solvent system.

Compound 3 may be converted to Compound 4 (2-((3S)-5-ethoxy-2-methoxy-1,3-dimethylindolin-3-yl)-N,N,N-trimethylethanaminium iodide) by methylation with methyl iodide.

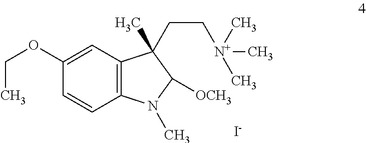 [SMILES: CCOc1ccc2c(c1)[C@](C)(CC[N+](C)(C)C)C(OC)N2C ; [I-]]

The methylation of 3 may be performed in an organic solvent, such as an ether. Non-limiting examples of ethers considered with the methods of the invention are diethyl ether or tetrahydrofuran. Progress of the reaction may be followed by a method such as TLC, HPLC or NMR. Once deemed sufficiently complete, the reaction mixture may be concentrated under vacuum to isolate the product. In a non-limiting embodiment, Compound 4 may be isolated in sufficient purity to be used as is in the next step. In another non-limiting embodiment, Compound 4 may be purified by any purification method known to one skilled in the art, such as but not limited to preparative HPLC, silica gel column chromatography or crystallization from a solvent system.

Compound 4 may be converted to Compound 5 ((3aS)-1-benzyl-5-ethoxy-3a,8-dimethyl-1,2,3,3a,8,8a-hexahydropyrrolo[2,3-b]indole) by treatment with benzylamine.

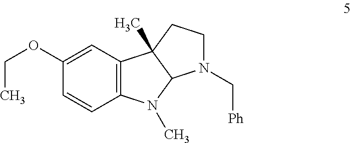 [SMILES: CCOc1ccc2c(c1)[C@]1(C)CCN(Cc3ccccc3)C1N2C]

Compound 4 may be treated with benzylamine in a polar organic solvent, such as but not limited to dimethylsulfoxide, dimethylformamide or N-methyl-pyrrolidinone. Progress of the reaction may be followed by a method such as TLC, HPLC or NMR. The product of the reaction may be isolated by addition of water to the reaction mixture and: (a) isolation of the resulting precipitate; or (b) extraction of the product with an organic solvent that is not water soluble, such as diethyl ether or tetrahydrofuran, and isolation of the product by concentration under vacuum. In a non-limiting embodiment, Compound 5 may be isolated in sufficient purity to be used as is in the next step. In another non-limiting embodiment, Compound 5 may be purified by any purification method known to one skilled in the art, such as but not limited to preparative HPLC, silica gel column chromatography or crystallization from a solvent system.

Compound 5 may be converted to Compound 6 ((3aS)-1-benzyl-5-ethoxy-3a-methyl-1,3,3a,8a-tetrahydropyrrolo[2,3-b]indole-8(2H)-carbaldehyde) by oxidation.

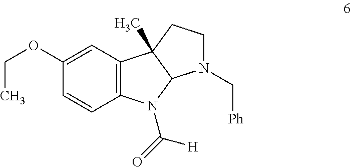 [SMILES: CCOc1ccc2c(c1)[C@]1(C)CCN(Cc3ccccc3)C1N2C=O]

Compound 5 may be treated with an oxidant that is reactive enough to oxidize the N-methyl group to a N-formyl group, yet mild enough not to further oxidize the N-formyl or any other functional group in Compound 5. No limiting examples of suitable oxidants for this transformation are pyridinium dichromate, silver nitrate/sodium persulfate, potassium permanganate, t-butyl hydrochlorite, N-bromosuccinimide, N-chlorosuccinimide and hydrogen peroxide. The reaction may be run in a solvent system comprising an aqueous solution and an organic solvent that is not soluble in the organic solution, such as diethyl ether or dichloromethane. The solvent system may further comprise an inorganic base, such as but not limited to sodium carbonate, sodium bicarbonate, sodium hydroxide or sodium phosphate. The reaction may be run at a temperature ranging from −20° C. to room temperature, and progress of the reaction may be followed by a method such as TLC, HPLC or NMR. Crude product may be isolated by filtering the reaction mixture and washing the solid residue with an organic solvent. In a non-limiting embodiment, Compound 6 may be isolated in sufficient purity to be used as is in the next step. In another non-limiting embodiment, Compound 6 may be purified by any purification method known to one skilled in the art, such as but not limited to preparative HPLC, silica gel column chromatography or crystallization from a solvent system.

Compound 6 may be converted to Compound 7 ((3aS)-1-benzyl-3a-methyl-1,2,3,3a,8,8a-hexahydropyrrolo[2,3-b]indol-5-ol) by using a reagent capable of simultaneous dealkylation and deformylation.

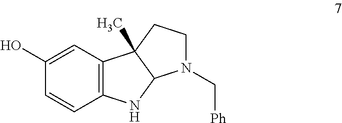 [SMILES: C[C@@]12CCN(Cc3ccccc3)C1Nc1ccc(O)cc12]

Compound 6 may be dissolved in an organic solvent, such as dichloromethane or diethyl ether, and treated with a dealkylating reagent, such as but not limited to boron tribromide, trimethylsilyl iodide, trimethylsilyl chloride, trifluoroboron etherate, tetrachlorosilane, aluminum tribromide, aluminum trichloride, ferric trichloride, or bromodimethylborane. The reaction may be run at a temperature ranging from −78° C. to room temperature, and progress of the reaction may be followed by a method such as TLC, HPLC or NMR. The reaction mixture may be quenched with an aqueous solution optionally comprising a dilute inorganic acid or an acidic inorganic salt. The product may separate as a solid upon the addition of the aqueous solution, in which case the product may be isolated by filtering the reaction mixture and washing the solid with water and appropriate organic solvents. In a non-limiting embodiment, Compound 7 may be isolated in sufficient purity to be used as is in the next step. In another non-limiting embodiment, Compound 7 may be purified by any purification method known to one skilled in the art, such as but not limited to preparative HPLC, silica gel column chromatography or crystallization from a solvent system.

Compound 7 may be converted to Compound 8 ((3aS)-tert-butyl 1-benzyl-5-hydroxy-3a-methyl-1,3,3a,8a-tetrahydropyrrolo[2,3-b]indole-8(2H)-carboxylate) by treatment with a BOC-protecting reagent, such as di-tert-butyl dicarbonate (also known as BOC anhydride).

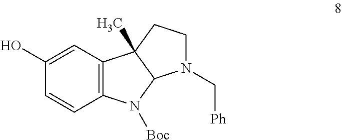 [SMILES: CC(C)(C)OC(=O)N1c2ccc(O)cc2[C@]2(C)CCN(Cc3ccccc3)C12]

Compound 7 may be dissolved in an organic solvent, such as but not limited to N,N-dimethylformamide, dimethyl sulfoxide or tetrahydrofuran, and treated with an aqueous solution of an inorganic base, such as but not limited to sodium carbonate, sodium bicarbonate, sodium hydroxide or sodium phosphate. The resulting system may be treated with BOC anhydride at a temperature ranging from −20° C. to 50° C., and progress of the reaction may be followed by a method such as TLC, HPLC or NMR. The reaction mixture may be worked up by adding water and an organic solvent that is not water soluble, such as diethyl ether, tetrahydrofuran or dichloromethane, to the reaction mixture. The product may be isolated from the organic layer by concentration under vacuum. In a non-limiting embodiment, Compound 8 may be isolated in sufficient purity to be used as is in the next step. In another non-limiting embodiment, Compound 8 may be purified by any purification method known to one skilled in the art, such as but not limited to preparative HPLC, silica gel column chromatography or crystallization from a solvent system.

Compound 8 may be converted to Compound 9 ((3aS)-tert-butyl 1-benzyl-5-(((4-isopropylphenyl)carbamoyl)oxy)-3a-methyl-1,3,3a,8a-tetrahydropyrrolo[2,3-b]indole-8(2H)-carboxylate).

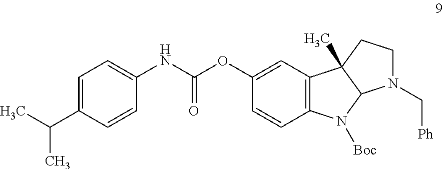 [SMILES: CC(C)c1ccc(NC(=O)Oc2ccc3c(c2)[C@]2(C)CCN(Cc4ccccc4)C2N3C(=O)OC(C)(C)C)cc1]

In one embodiment, Compound 9 may be prepared by treating Compound 8 with isopropyl isocyanate in an organic solvent, such as but not limited to dichloromethane, tetrahydrofuran or diethyl ether. The reaction may be run in the presence of an organic base, such as sodium ethoxide, sodium methoxide, sodium hydride, potassium hydride and the like. The reaction may run at a temperature ranging from −50° C. to 50° C., and progress of the reaction may be followed by a method such as TLC, HPLC or NMR.

In another embodiment, Compound 9 may be prepared in a two-step procedure from Compound 8. In the first step, Compound 8 may be treated with phosgene, diphosgene, triphosgene, carbonyldiimidazole or para-nitrophenyl-chloroformate in an organic solvent, such as but not limited to dichloromethane, tetrahydrofuran or diethyl ether, to form the corresponding chloroformate (in the case of phosgene, diphosgene, or triphosgene), imidazolyl carbonyl derivative (in the case of carbonyldiimidazole) or para-nitrophenyl carbonate (in the case of p-nitrophenyl-chloroformate). The product of this reaction may then be treated with para-isopropylaniline in an organic solvent, such as but not limited to dichloromethane, tetrahydrofuran or diethyl ether. Either reaction may run at a temperature ranging from −50° C. to 80° C., and progress of either reaction may be followed by a method such as TLC, HPLC or NMR.

The reaction may be quenched with an aqueous solution optionally comprising a basic inorganic salt or inorganic base. The product may be extracted with an organic solvent such as dichloromethane or diethyl ether, and isolated by concentration under vacuum. In a non-limiting embodiment, Compound 9 may be isolated in sufficient purity to be used as is in the next step. In another non-limiting embodiment, Compound 9 may be purified by any purification method known to one skilled in the art, such as but not limited to preparative HPLC, silica gel column chromatography or crystallization from a solvent system.

Compound 9 may be converted to Compound 10 ((3aS)-tert-butyl 5-(((4-isopropylphenyl)carbamoyl)oxy)-3a-methyl-1,3,3a,8a-tetrahydropyrrolo[2,3-b]indole-8(2H)-carboxylate) by hydrogenation.

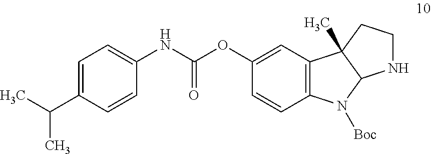 [SMILES: CC(C)c1ccc(NC(=O)Oc2ccc3c(c2)[C@]2(C)CCNC2N3C(=O)OC(C)(C)C)cc1]

In one embodiment, Compound 9 may be reacted with hydrogen gas in the presence of a hydrogenation catalyst, such as but not limited to palladium metal on carbon, palladium dihydroxide on carbon, palladium on barium sulfate or platinum metal on carbon. In another embodiment, Compound 9 may be reacted with a hydrogen transfer reagent in the presence of a catalyst, such as but not limited to ammonium formate or cyclohexadiene in the presence of palladium metal on carbon. The reaction may run at a temperature ranging from −20° C. to 80° C., and progress of the reaction may be followed by a method such as TLC, HPLC or NMR. The product of the reaction may be isolated by filtration and concentration of the filtrate under vacuum. In a non-limiting embodiment, Compound 10 may be isolated in sufficient purity to be used as is in the next step. In another non-limiting embodiment, Compound 10 may be purified by any purification method known to one skilled in the art, such as but not limited to preparative HPLC, silica gel column chromatography or crystallization from a solvent system.

Compound 10 may be converted to Compound 1 ((3aS)-3a-methyl-1,2,3,3a,8,8a-hexahydropyrrolo[2,3-b]indol-5-yl (4-isopropylphenyl)carbamate) by acidic hydrolysis.

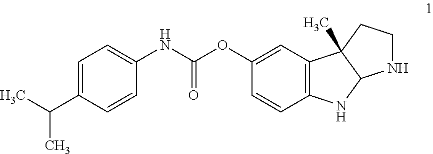 [SMILES: CC(C)c1ccc(NC(=O)Oc2ccc3c(c2)[C@]2(C)CCNC2N3)cc1]

Compound 10 may be deprotected by treatment with an acid, such as but not limited to trifluoroacetic acid in dichloromethane, trifluoroacetic acid in water, aqueous hydrochloric acid or hydrogen chloride solution in tetrahydrofuran. Progress of the reaction may be followed by a method such as LC-MS, TLC, HPLC or NMR.

In a non-limiting embodiment, Compound 10 (1 volume) may be dissolved in dichloromethane (in a non-limiting embodiment, 10 volumes), and the solution may be treated with trifluoroacetic acid (in a non-limiting embodiment, 16 equivalents) at 0-5° C. under stirring. The system may be stirred for 4 hours at 25-30° C., and the volatiles in the mixture may be distilled, for example, at 44° C. under 2 mm pressure. The resulting system may be treated with one or more of the following solutions: aqueous saturated sodium bicarbonate solution (in a non-limiting embodiment, 16 volumes), dichloromethane (in a non-limiting embodiment, 5.8 volumes), brine (in a non-limiting embodiment, 15.7 volumes) and sodium sulfate (in a non-limiting embodiment, 0.6 w/w). The system may be allowed to separate phases, and the organic phase may be reserved and concentrated, for example at 44° C. under 600 mm pressure. The resulting residue may be dried, yielding the product.

Compound 1 may be converted to Compound 11 (the tartrate salt of (3aS)-3a-methyl-1,2,3,3a,8,8a-hexahydropyrrolo[2,3-b]indol-5-yl-(4-isopropylphenyl) carbamate) by treatment with tartaric acid. The representation of compound 11 illustrated below does not imply any particular ratio of the Compound 11 and tartaric acid in the composition.

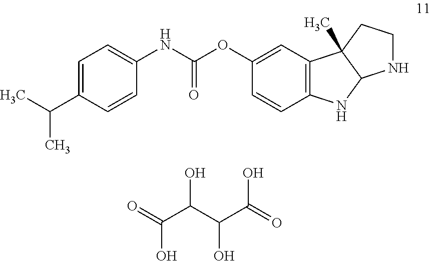 [SMILES: CC(C)c1ccc(NC(=O)Oc2ccc3c(c2)[C@]2(C)CCNC2N3)cc1 ; O=C(O)C(O)C(O)C(=O)O]

It is understood that the compositions and methods of the present invention contemplate and include any salt comprising an acid and Compound 1, wherein the acid and Compound 1 may be present in any particular ratio, wherein the salt is stable (i.e., the salt that does not decompose spontaneously under normal temperature and pressure conditions).

Compound 1 may be treated with a solution of tartaric acid in an organic solvent, such as an alcohol. Non-limiting examples of alcohols useful within the methods of the invention are methanol, ethanol, 1-propanol, isopropanol, tert-butanol, n-butanol or sec-butanol.

In a non-limiting embodiment, Compound 1 may be dissolved in isopropanol (in a non-limiting embodiment, 5 volumes) and treated with a solution of L-tartaric acid in isopropanol (in a non-limiting embodiment, 5 volumes) at 55° C. under stirring. The resulting system may be stirred at 45-75° C. for one hour, and then filtered. The solid isolated may be washed with isopropanol (in a non-limiting embodiment, 3 volumes) and then dried at 40° C. for 4 hours. The residue may be stirred with a solvent system comprising DMSO (in a non-limiting embodiment, 10 volumes) and water (in a non-limiting embodiment, 12 volumes), at 0-10° C. for 1 hour, and then filtered. The residue may be dried at 44° C. for 96 hours. The residue may be stirred in acetonitrile (in a non-limiting embodiment, 11 volumes) at 25-30° C. for 3 hours, and then filtered. The residue may be freeze-dried or spray-dried to afford Compound 11.

### Salts

The compounds described herein may form salts with acids or bases, and such salts are included in the present invention. In one embodiment, the salts are pharmaceutically acceptable salts. The term “salts” embraces addition salts of free acids or free bases that are compounds of the invention. The ratio between the acid and the base in the salt may be any positive number and is not necessary a ratio between integers (i.e., the salt contemplated within the compositions and methods of the invention may be stoichiometric or non-stoichiometric).

The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding compound by reacting, for example, the appropriate acid or base with the compound.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all remaining starting materials were obtained from commercial suppliers and used without purification.

### Preparative Example 1

### (3S)-3-(2-(dimethylamino)ethyl)-5-ethoxy-1,3-dimethylindolin-2-ol, 3

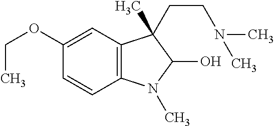 [SMILES: CCOc1ccc2c(c1)[C@](C)(CCN(C)C)C(O)N2C]

(−)-Eserethole 2 (1,000 g; 4.059 mmol) was dissolved in diethyl ether at 40° C. in a 20.0 liter three-neck round bottom flask fitted with a calcium chloride guard tube and a mechanical overhead stirrer. The resulting clear light yellow solution was cooled to 20° C.

Methyl iodide (1,020 mL; 16.230 mmol) was then added to the solution at 20° C. dropwise through a dropping funnel with constant stirring. A white precipitate gradually formed in the reaction mixture. Stirring was continued at 20° C.

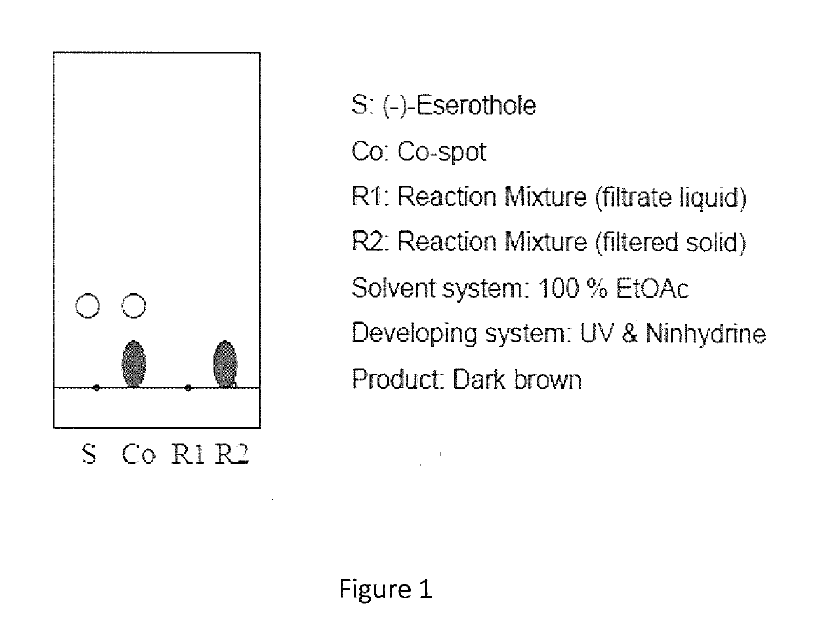

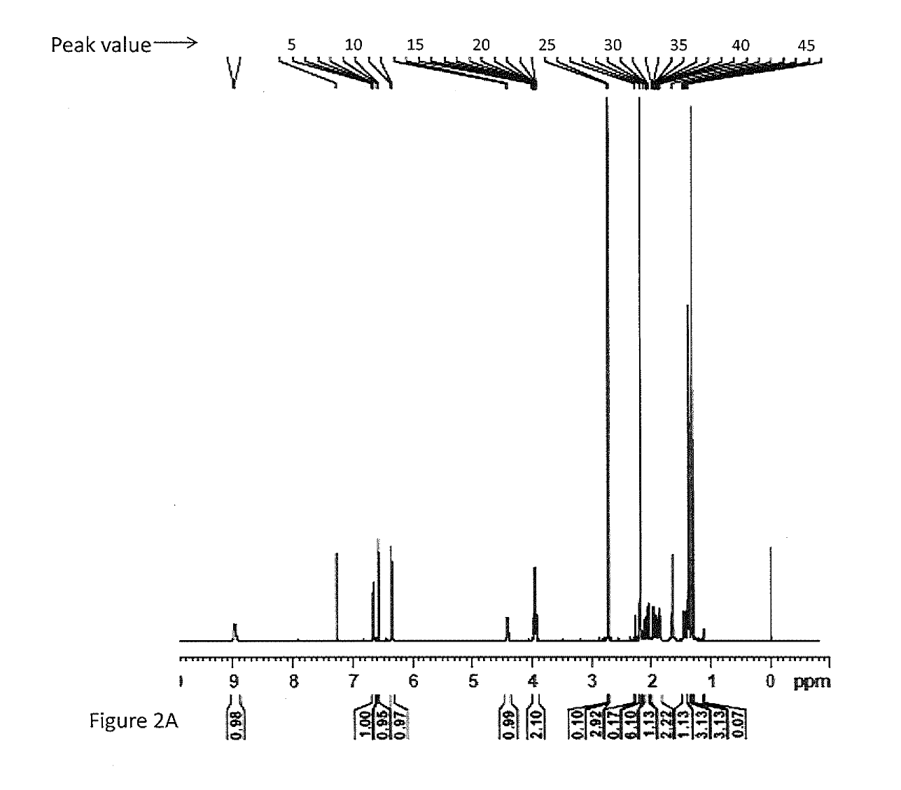

In the TLC spotting method, a 1 mL aliquot of the reaction mixture was removed by dropper and filtered. The filtered solid and filtrate liquid were analyzed by TLC separately. Monitoring of the reaction by TLC indicated ˜70 conversion of starting material to new spot (spot R1, FIG. 1). Further methyl iodide (500 mL; 7,961.1 mmol) was added to the solution dropwise with constant stirring. Monitoring of the reaction by TLC showed complete conversion of starting material to a new polar spot (spot R2, FIG. 1).

Diethyl ether was distilled from the reaction mixture under reduced pressure at 38° C. The resulting light yellow solid was then dissolved in water in a 20.0 liter three-neck round bottom flask fitted with a mechanical overhead stirrer. A clear light yellow solution was formed. A solution of sodium hydroxide (1,623.7 g; 70.596 mmol) in water (4.0 liters) was added dropwise through a dropping funnel to the reaction mixture over a period of 35 minutes at 30-35° C. with constant stirring. A light yellow solid formed in the reaction mixture. Stirring was then continued at 30-35° C. overnight.

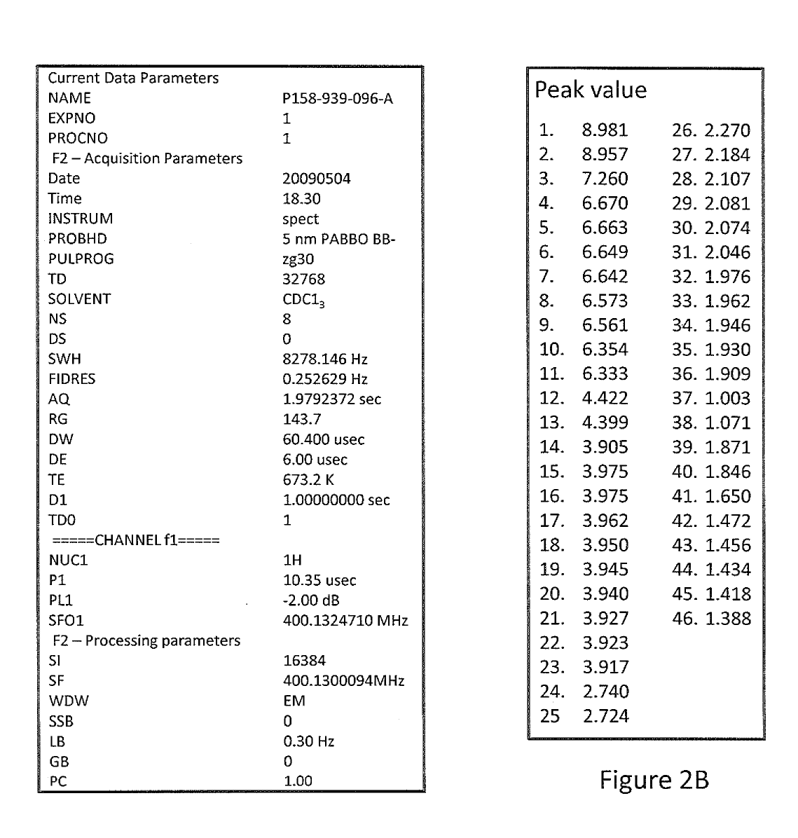

The thick light yellow solid formed in the reaction mixture was filtered through a Buckner funnel. The filtered solid was then washed with water (4.0 liters) and dried under vacuum at 35° C. to afford 1031.0 g (91.23%) of a light yellow solid. A ¹H NMR analysis (CDCl₃) of this material (FIG. 2) confirmed the formation of desired (3).

### Preparative Example 2

### 2-((3S)-5-ethoxy-2-methoxy-1,3-dimethylindolin-3-yl)-N,N,N-trimethylethanaminium iodide, 4

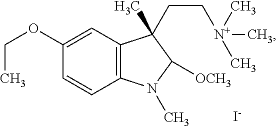 [SMILES: CCOc1ccc2c(c1)[C@](C)(CC[N+](C)(C)C)C(OC)N2C ; [I-]]

Compound 3 (1,030 g; 3.699 mmol) was dissolved in diethyl ether (10.3 liters) at 30° C. in a 20.0 liter four-neck round bottom flask fitted with a mechanical overhead stirrer and a calcium chloride guard tube. The solution was then cooled to 15-20° C. A clear solution was formed.

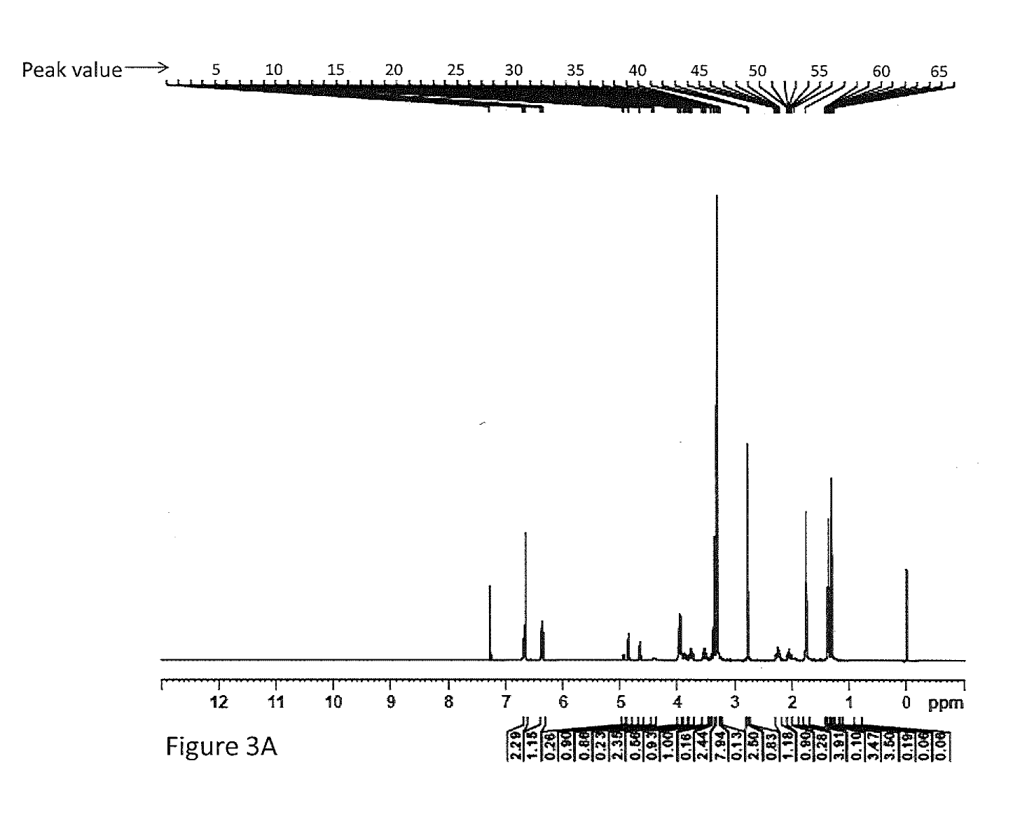

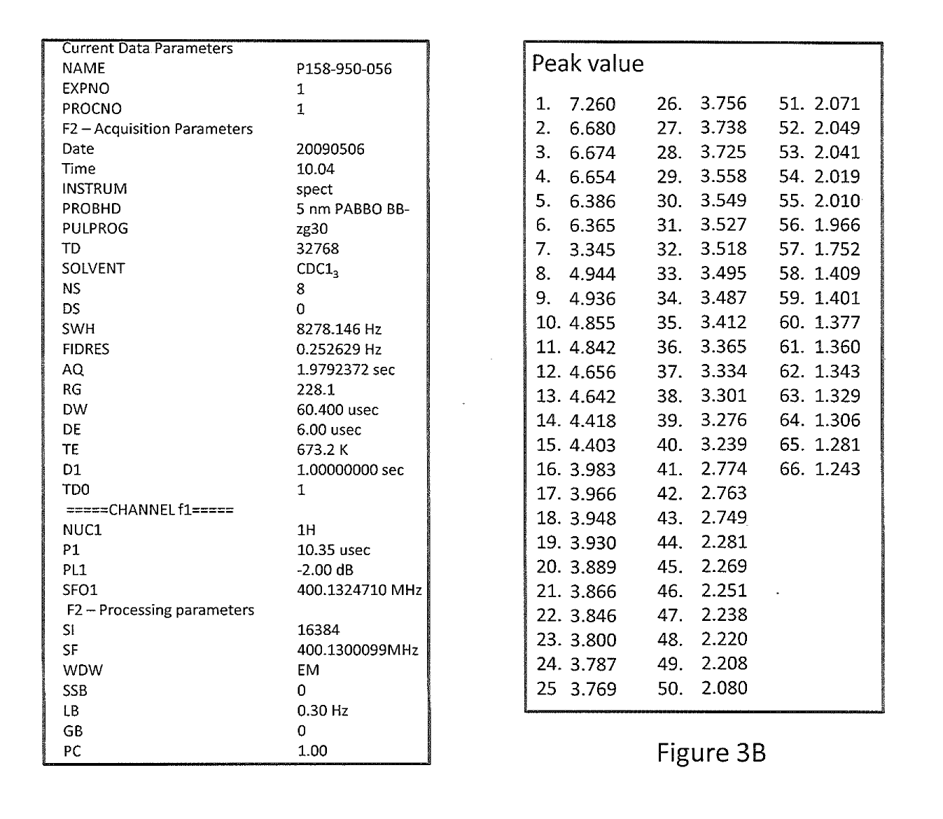

Methyl iodide (1,162 liters; 18.499 mmol) was added to the solution dropwise over a period of 80.0 minutes at 15-20° C. with constant stirring. A thick white solid was gradually formed in the reaction mixture, Stirring was continued. The white solid precipitate was filtered through a Buckner funnel. This filtered solid was washed with diethyl ether (2×1.25 liters). The filtered solid was dried at 30° C. under reduced pressure to afford 1550.0 g white solid powder (96.45%). A ¹H NMR analysis of this solid in CDCl₃ (FIG. 3) confirmed the formation of desired 4.

### Preparative Example 3

### (3aS)-1-benzyl-5-ethoxy-3a,8-d methyl-1,2,3,3a,8,8a-hexahydropyrrolo[2,3-b]indole, 5

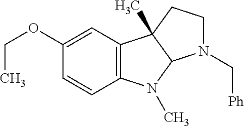 [SMILES: CCOc1ccc2c(c1)[C@]1(C)CCN(Cc3ccccc3)C1N2C]

Compound 4 (768.0 g; 1.768 mmol) was placed in a 10.0 liter three-neck round bottom flask fitted with a condenser and a mechanical overhead stirrer, placed over an oil bath. Dimethyl sulfoxide (2,304 mL) was added to the solid with constant stirring at 30° C. A clear solution was formed.

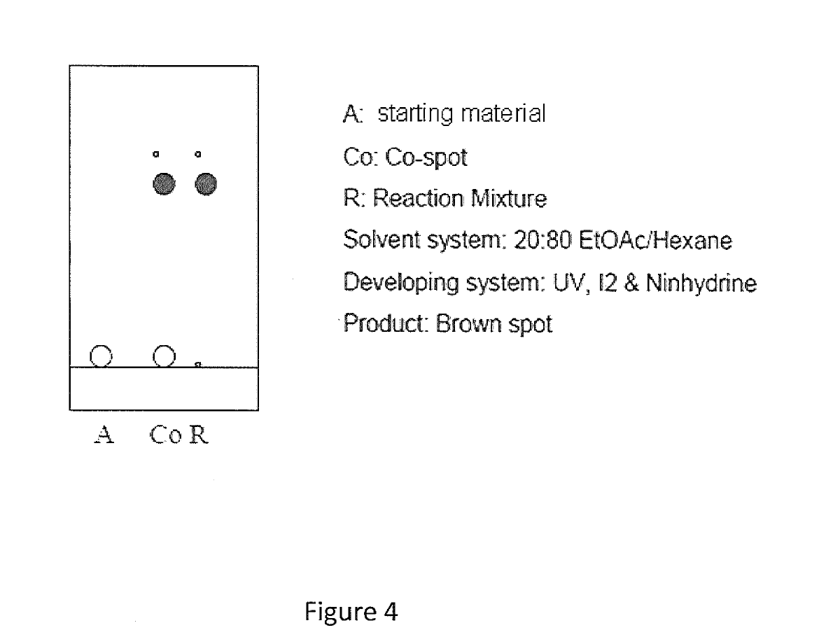

Benzyl amine (289.27 mL; 2.650 mmol) was added to the solution at 30° C. The reaction mixture was then heated to 114° C. The reaction was monitored by TLC, showing complete conversion of starting material to a new spot (FIG. 4). In the TLC spotting method, a ˜1.0 mL aliquot of the reaction mixture was quenched in ˜1.0 mL water in a vial. Ethyl ether (1 mL) was added and the system was shaken thoroughly. The upper organic layer was used for TLC spotting.

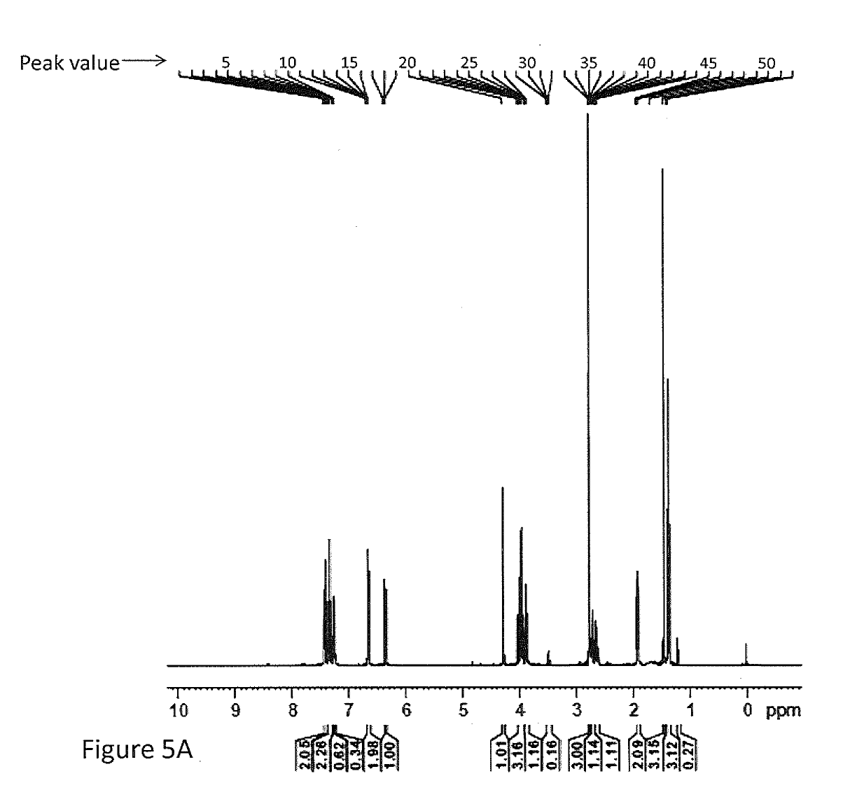

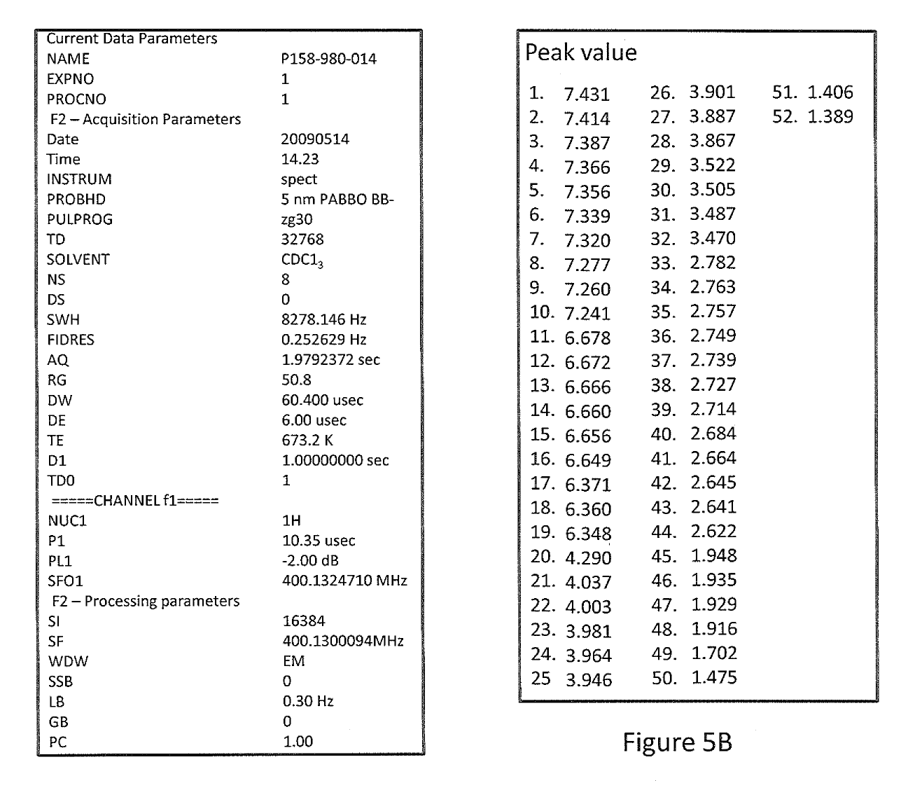

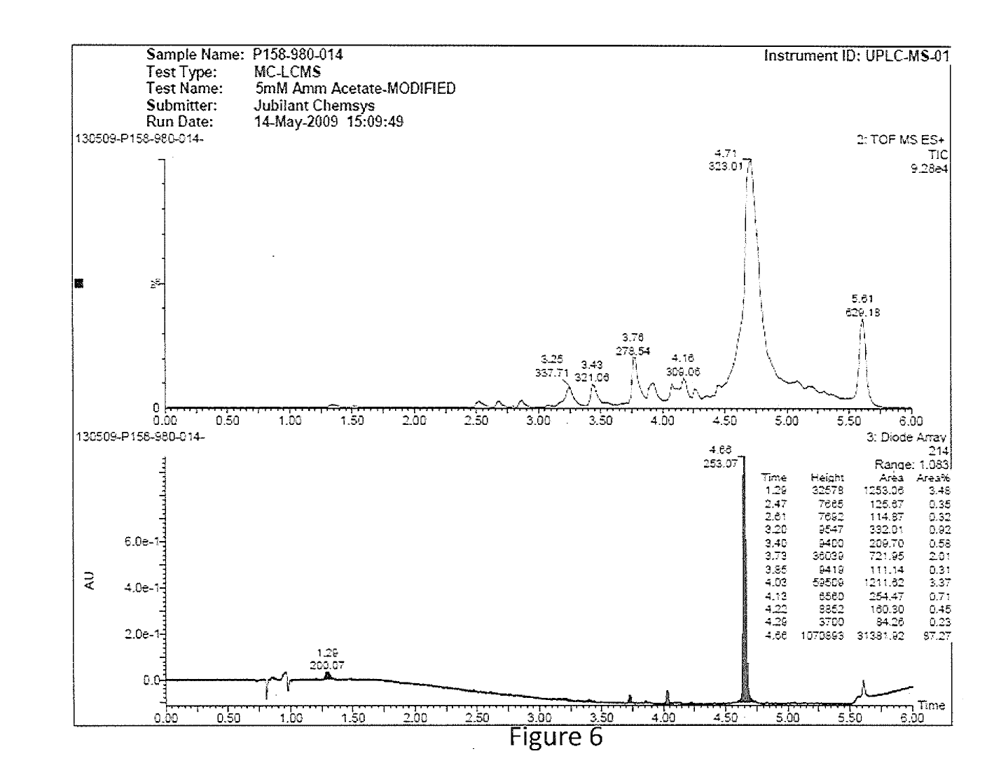

For the work-up, heating of the reaction mixture was stopped and the reaction mixture was cooled to 30° C. Water (3.5 liters) was added to the reaction mixture with constant stirring. A yellow solid was formed, and the system was extracted with diethyl ether (3×2.0 liters). The total ether layer was then washed with water (1.5 liters) and brine (1.5 liters), dried over sodium sulfate, and concentrated under vacuum to afford 524.0 g brown crystalline material. A NMR analysis of this solid (FIG. 5) confirmed the formation of desired 5 (91.9% yield, with a purity of 87.27% by LC-MS). An illustrative LC-MS trace for the product is reproduced in FIG. 6.

### Preparative Example 4

### (3aS)-1-benzyl-5-ethoxy-3a-methyl-1,3,3a,8a-tetrahydropyrrolo[2,3-b]indole-8(2H)-carbaldehyde, 6

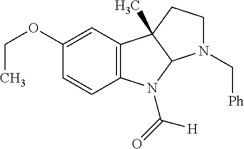 [SMILES: CCOc1ccc2c(c1)[C@]1(C)CCN(Cc3ccccc3)C1N2C=O]

Compound 5 (550.0 g, 1.700 mmol) was dissolved in dichloromethane (16,500 mL) at 30° C. in a 20.0 liter four-neck round bottom flask fitted with a mechanical overhead stirrer and a calcium chloride guard tube, placed over a plastic bucket. A brown clear solution was formed.

Sodium bicarbonate (501.48 g; 5.970 mmol) was added to it at 30° C. with constant stirring. The system was cooled to 0° C. Pyridinium dichromate (1,604.2 g; 4.260 mmol) was then added to it portionwise over a period of 45 minutes at 0° C. with constant stirring. The reaction mixture was gradually warmed to 18° C., and stirring was continued maintaining the temperature 18° C.

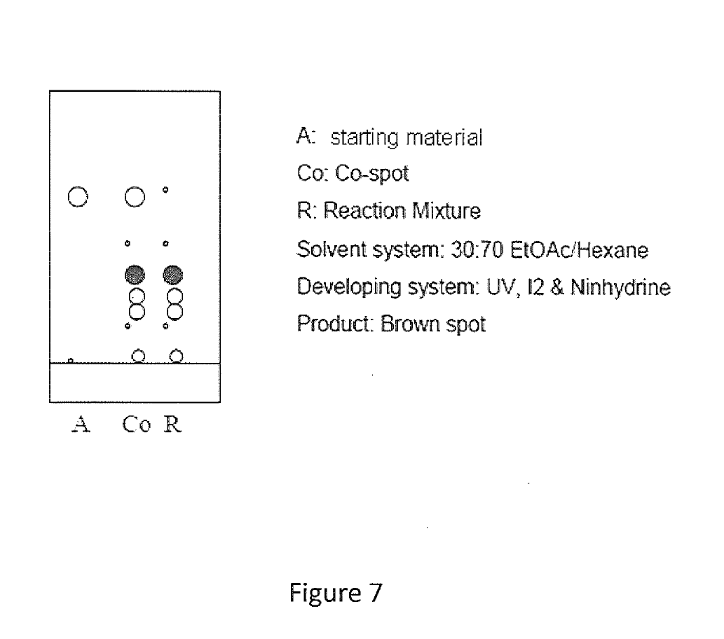

TLC monitoring of the reaction showed ˜90% conversion of starting to new spots (FIG. 7). In the TLC spotting method, ˜1.0 mL reaction mixture was aliquotted and the upper organic layer was used for TLC spotting.

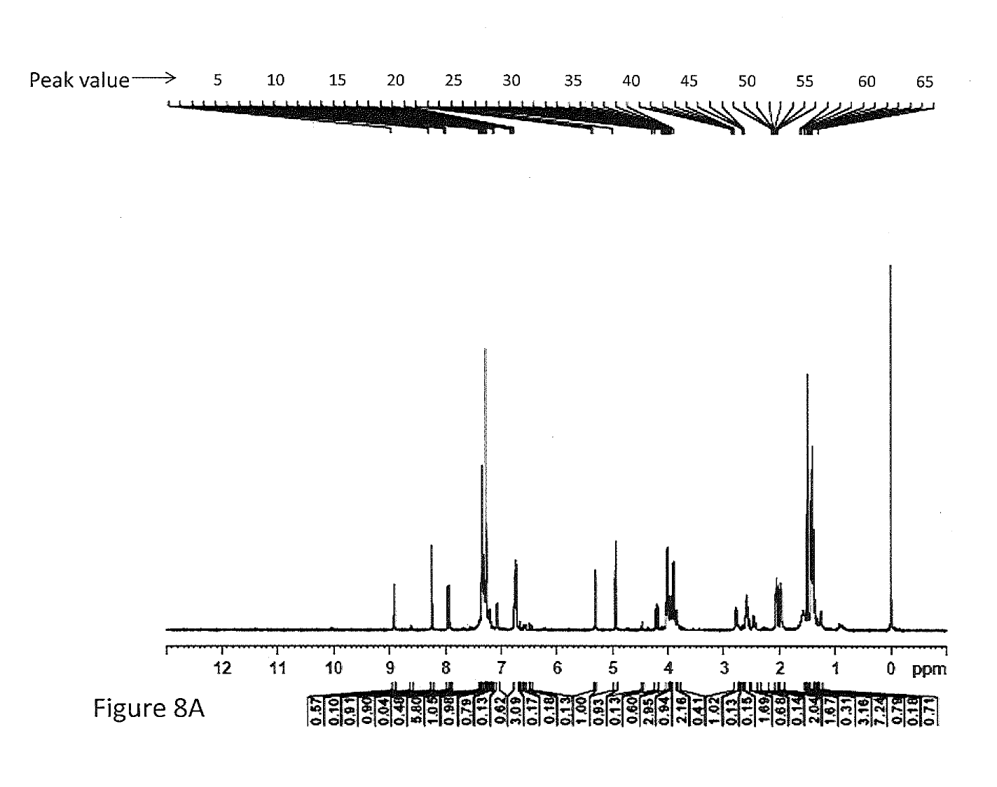

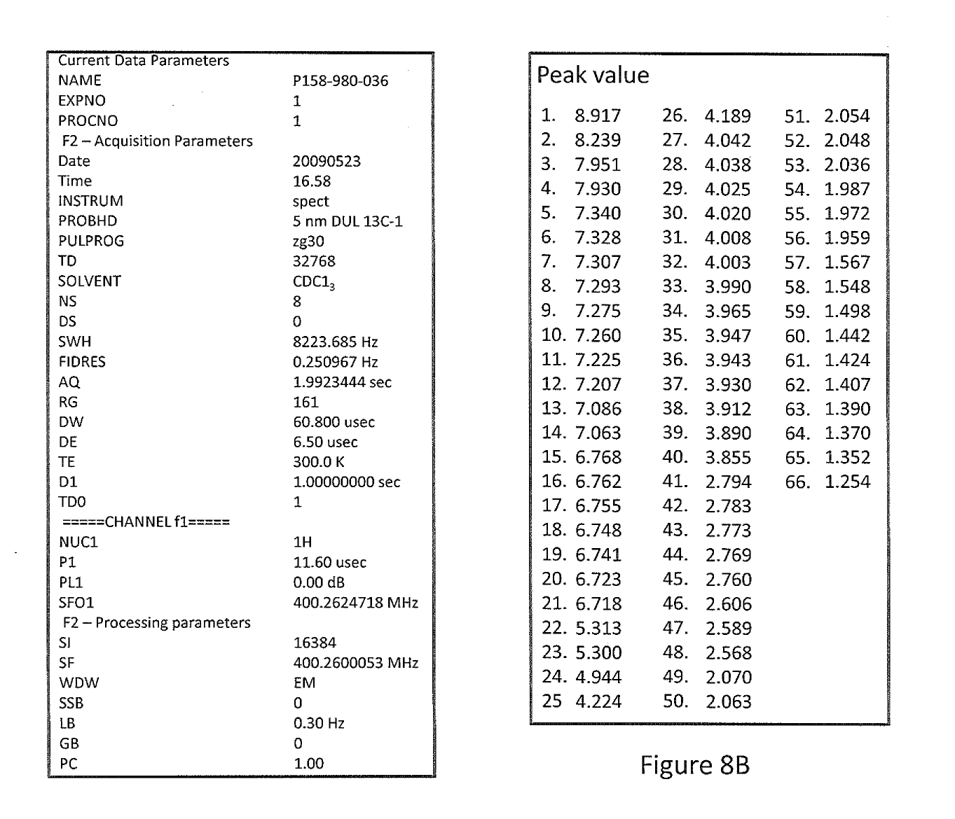

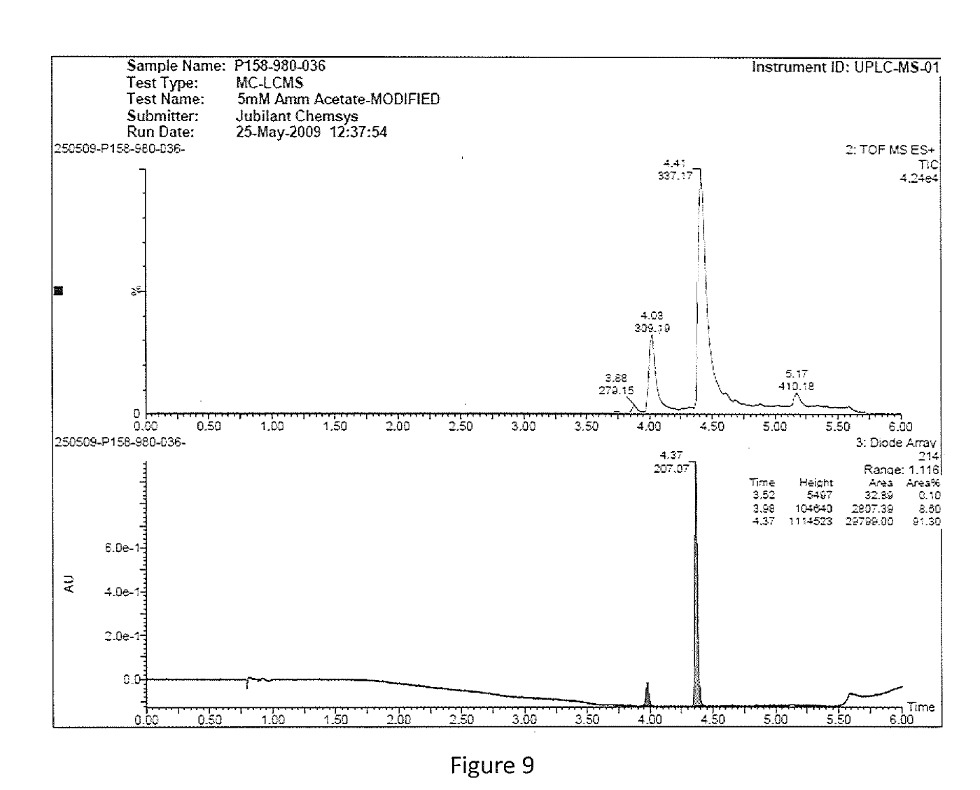

The reaction mixture was filtered through Buckner funnel. The filtered solid was washed with dichloromethane (5×1.0 liter). The total filtrate liquid was concentrated under vacuum at 40° C. to afford 756.0 g black semi solid compound. This crude material was purified by column chromatography using 100-200 mesh silica gel and ethyl acetate-hexane as solvent system. The desired brown spot was eluted at 14:86% ethyl acetate-hexane to 18:82% ethyl acetate-hexane to afford 205.0 g brown liquid. A ¹H NMR analysis of this product (FIG. 8) in CDCl₃ confirmed the formation of desired 6. The yield was 35.7%, and the purity was 91.3% by LC-MS (FIG. 9).

### Preparative Example 5

### (3aS)-1-benzyl-3a-methyl-1,2,3,3a,8,8a-hexahydropyrrolo[2,3-b]indol-5-ol, 7

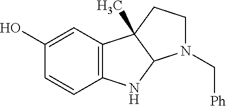 [SMILES: C[C@@]12CCN(Cc3ccccc3)C1Nc1ccc(O)cc12]

Compound 6 (100.0 g, 297.0 mmol) was dissolved in dichloromethane (1.0 liter) in a 5.0 lit three-neck round bottom flask fitted with a mechanical overhead stirrer and a calcium chloride guard tube at 30° C. The resulting brown clear solution was cooled to 0° C.

Boron tribromide (83.25 mL, 891.0 mmol) was added to the solution dropwise maintaining the temperature at 0° C. White fumes were observed. Once the addition of boron tribromide was completed, the system was gradually warmed to 20° C. with constant stirring.

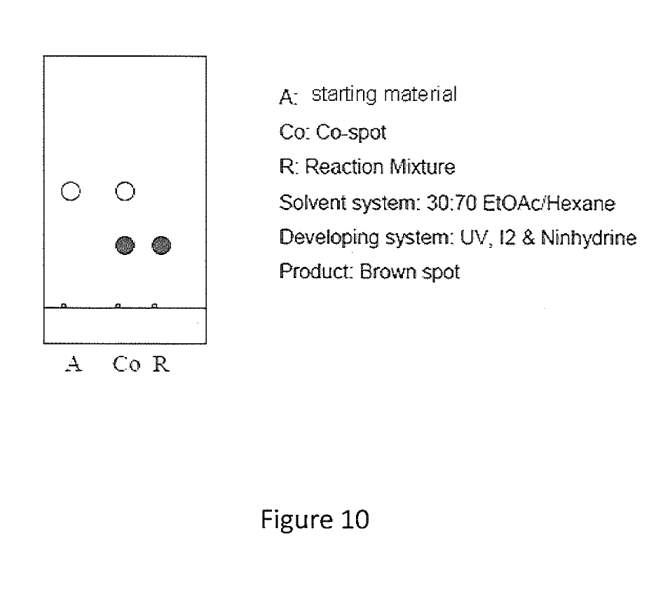

The reaction was monitored by TLC (FIG. 10), showed complete conversion of starting to new spot. In the TLC spotting method, ˜0.5 mL reaction mixture was quenched it over ˜1.0 g crushed ice. EtOAc (1.0 mL) was added to the system and shaken thoroughly. The upper organic layer was used for TLC spotting.

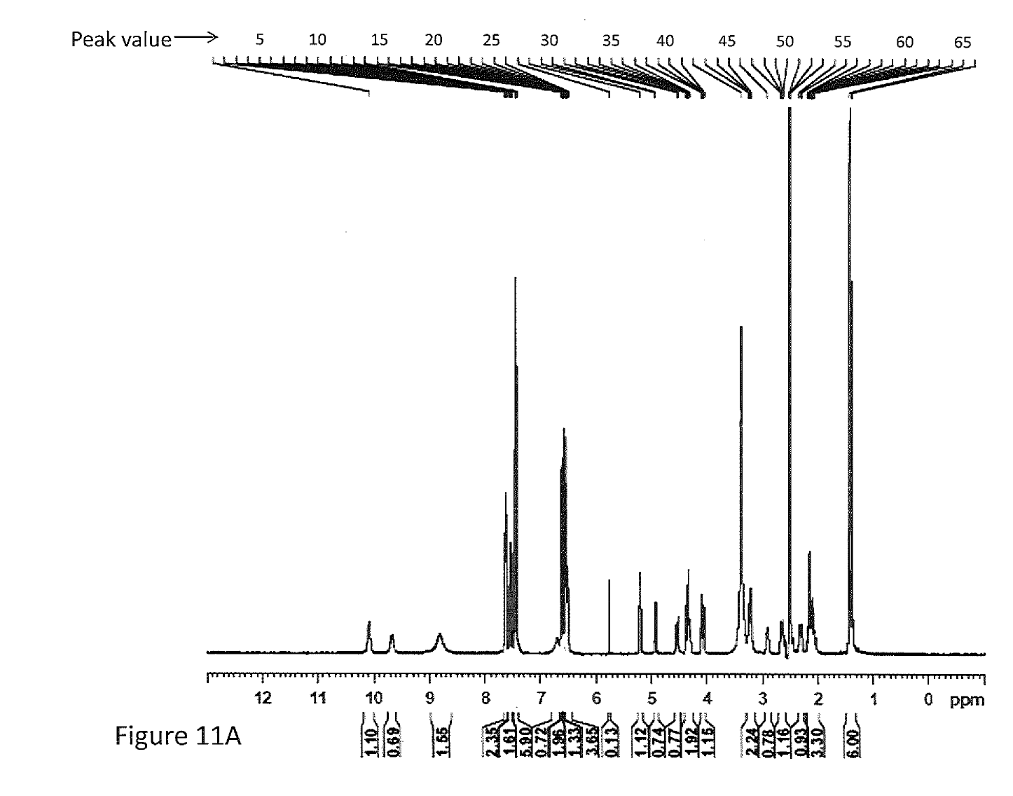

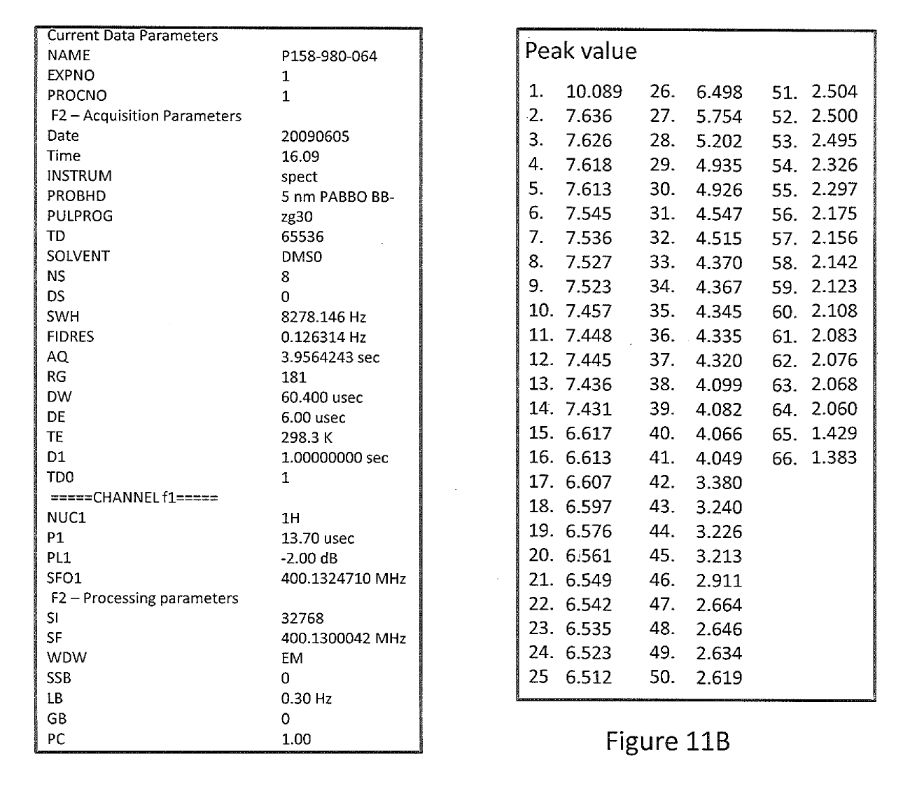

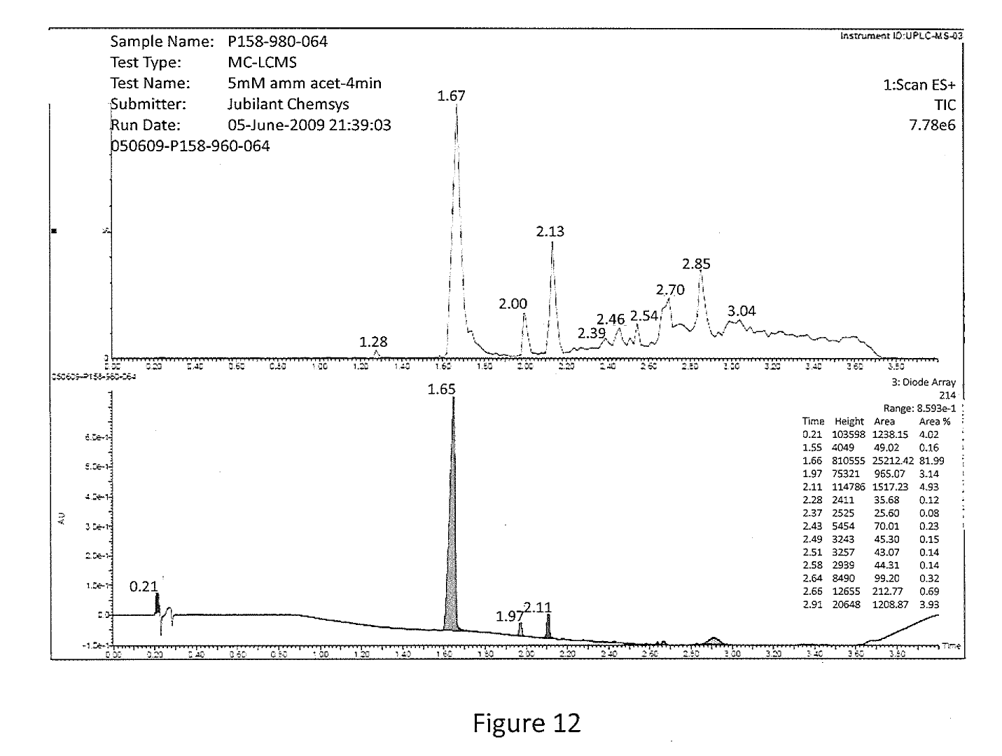

The reaction mixture was cooled to 0° C. The reaction was then quenched by dropwise addition of cold water (˜150.0 mL). Further 200.0 mL cold water were added to the system with constant stirring. 500 mL 4 N HCl were added to the system with constant stirring at 25° C., followed by stirring at 30° C. overnight. Further 1.2 liters water were added to the system and stirred for another 30 minutes. A yellowish solid formed in the system was filtered through a Buckner funnel and washed with water (300 mL) and dichloromethane (300 mL). This solid residue was dried at 30° C. under reduced pressure to afford 79.0 g of yellowish solid product. A ¹H NMR analysis (FIG. 11) of this solid confirmed the formation of product 7. The yield was 94.8%, and the purity was 81.95% by LC-MS (FIG. 12).

### Preparative Example 6

### (3aS)-tert-butyl 1-benzyl-5-hydroxy-3a-methyl-1,3,3a,8a-tetrahydropyrrolo[2,3-b]indole-8(2H)-carboxylate, 8

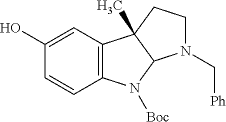 [SMILES: CC(C)(C)OC(=O)N1c2ccc(O)cc2[C@]2(C)CCN(Cc3ccccc3)C12]

Compound 7 (165.0 g; 0.588 mol) was dissolved in DMF (N,N-dimethylformamide; 750 mL) in a 5.0 liter three-neck round bottom flask fitted with a mechanical overhead stirrer and a calcium chloride guard tube at 30° C. A brown clear solution was formed.

Potassium carbonate (89.39 g; 0.646 mol) was added to the solution portionwise over a period of 10 minute at 30° C. The reaction mixture was stirred for 45 minutes. Di-tert-butyl dicarbonate (Boc anhydride; 122.08 g; 0.559 mol) was added to the system dropwise over a period of 30 minutes maintaining the temperature at 30° C. The reaction mass was stirred for 4 hours.

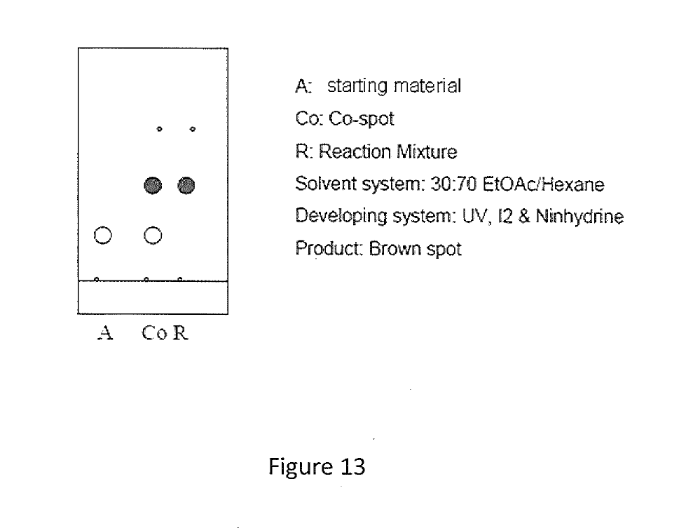

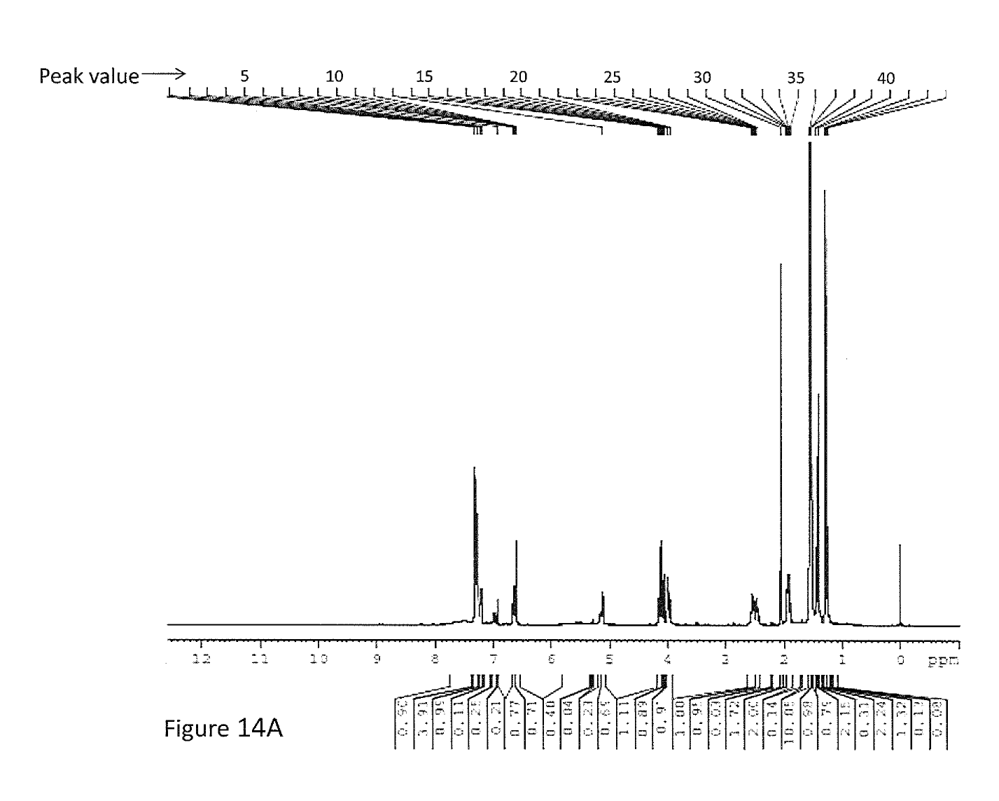

The reaction was monitored by TLC, showing complete conversion of starting to new spot (FIG. 13). In the TLC spotting method, ˜0.5 mL reaction mixture was transferred to a vial. 1.0 mL water and 1.0 mL diethyl ether were added to the reaction mixture and shook thoroughly. The upper organic layer was used for TLC spotting.

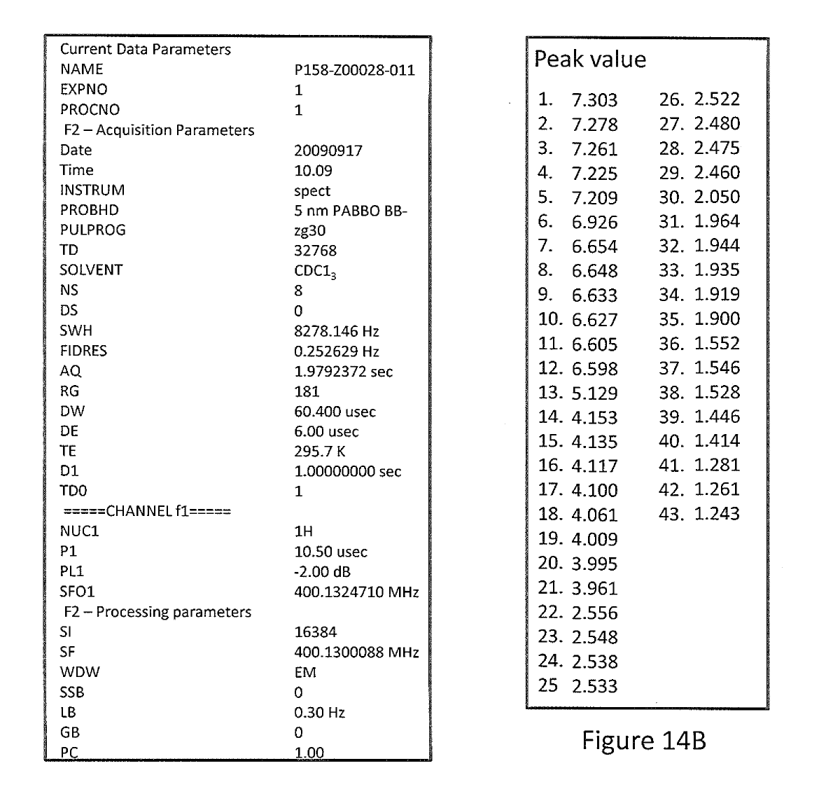

Water (2.0 liters) was added to the reaction mass and stirred for 10 minutes. A white suspension was observed in the reaction mass. Diethyl ether (10.0 liters) was added to the system and stirred the reaction mass for further 10 minutes. The upper organic layer was collected, and the aqueous layer was again extracted by diethyl ether (5.0 liters). The combined organic layer was washed with water (5.0 liters) and brine (5.0 liters), dried over Na₂SO₄ and then concentrated at 30° C. under reduced pressure to afford 175.0 g solid, A ¹H NMR analysis of this solid (FIG. 14) confirmed the formation of desired 8. The yield was 176.0 g (88.9%) of brown puffy solid.

### Preparative Example 7

### (3aS)-tert-butyl 1-benzyl-5-(((4-isopropylphenyl)carbamoyl)oxy)-3a-methyl-1,3,3a,8a-tetrahydropyrrolo[2,3-b]indole-8(2H)-carboxylate, 9

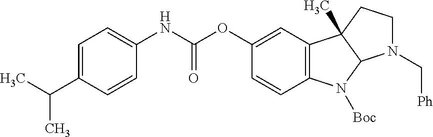 [SMILES: CC(C)c1ccc(NC(=O)Oc2ccc3c(c2)[C@]2(C)CCN(Cc4ccccc4)C2N3C(=O)OC(C)(C)C)cc1]

Compound 8 (176.0 g, 0.463 mol) was dissolved in tetrahydrofuran (5.2 liters) in a 10.0-liter three-neck round bottom flask fitted with a mechanical overhead stirrer and a calcium chloride guard tube at 30° C. under nitrogen atmosphere. A brown clear solution was formed. The reaction mixture was cooled to 0° C.

Sodium ethoxide (29.18 g, 0.449 mol) was added to the system portionwise over a period of 10.0 minutes maintaining the temperature of 0° C. Stirring was then continued for 30 minutes at 0° C. 1-Isocyanato-4-isopropylbenzene (74.61 g, 0.449 mol) was added to the system dropwise over a period of 30 minutes maintaining the temperature of 0° C. Once the addition of 1-isocyanato-4-isopropylbenzene was completed, the reaction mixture was warmed to 30° C. and stirred for 2 hrs at 30° C.

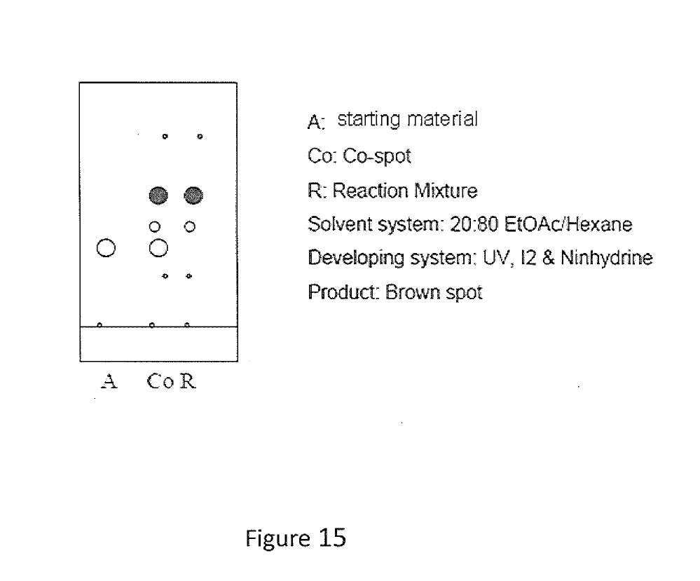

TLC monitoring of the reaction showed complete conversion of starting to new spots (FIG. 15). In the TLC spotting method, ˜0.5 mL reaction mixture was added to a vial, along with water (1.0 mL) and diethyl ether (1.0 mL) under thorough agitation. The upper organic layer was used for TLC spotting.

The reaction mixture was quenched by dropwise addition of cold water (˜150.0 mL). Further 2,000 mL cold water were then added to the system with constant stirring. Diethyl ether (10.0 liters) was added to it and stirred for further 30 minutes. The upper organic layer was collected, and the aqueous layer was again extracted by diethyl ether (2×2.5 liters). The combined organic layers were washed with water (3.0 liters) and brine (5.0 liters), dried over Na₂SO₄ and concentrated at 30° C. under reduced pressure to afford 253.0 g crude (101%) product.

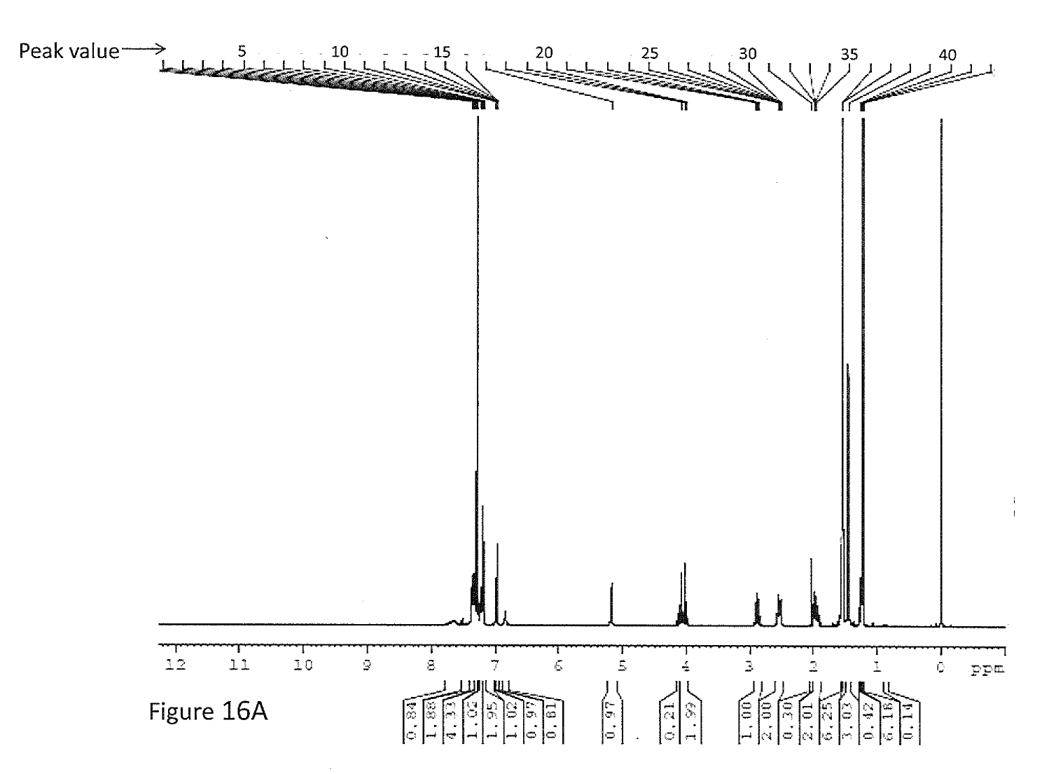

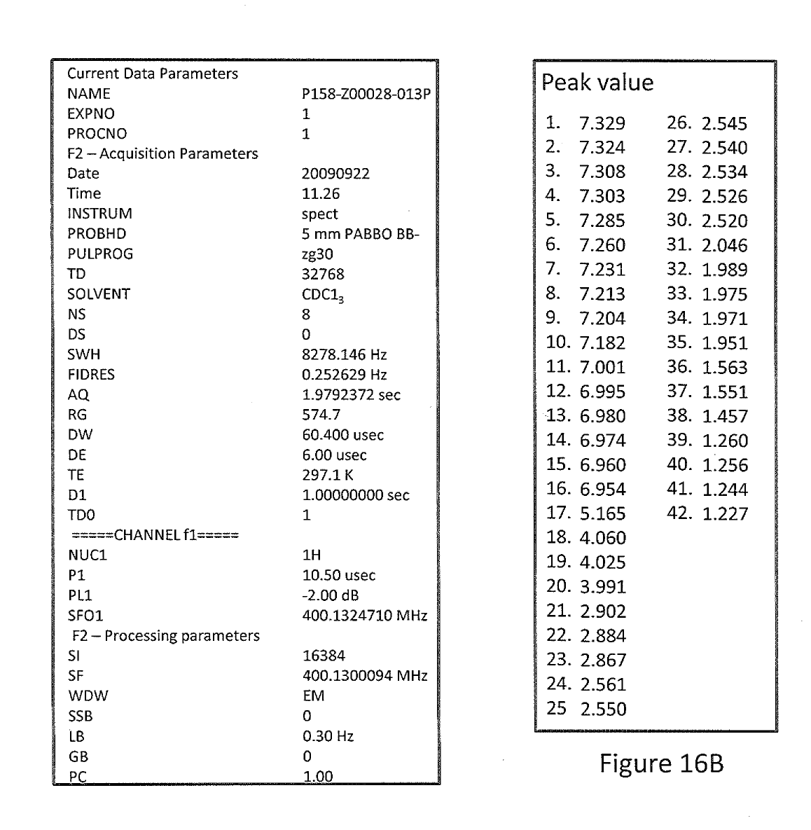

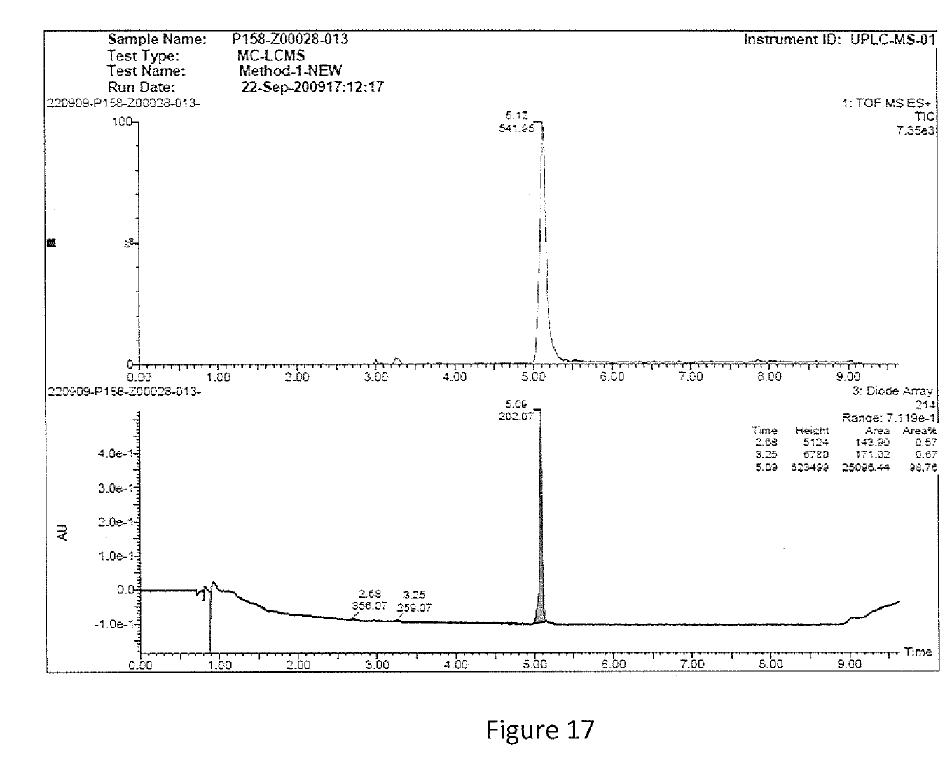

The crude product was purified by column chromatography using 100-200 mesh silica and ethyl acetate-hexane as an eluent. The desired fraction was eluted at 5:95 ethyl acetate-hexane to afford 150.0 g yellowish white-colored pure product. A ¹H NMR analysis of this fraction (FIG. 16) confirmed the formation of pure desired 9. The yield was 60%, and the purity was 98.27% by LC-MS (FIG. 17).

### Preparative Example 8

### (3aS)-tert-butyl 5-(((4-isopropylphenyl)carbamoyl)oxy)-3a-methyl-1,3,3a,8a-tetrahydropyrrolo[2,3-b]indole-8(2H)-carboxylate, 10

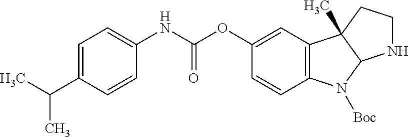 [SMILES: CC(C)c1ccc(NC(=O)Oc2ccc3c(c2)[C@]2(C)CCNC2N3C(=O)OC(C)(C)C)cc1]

Compound 9 (5.0 g, 9.24 mmole) was dissolved in ethanol (50.0 mL) in a 100.0 mL two-neck round bottom flask under nitrogen atmosphere. A clear yellowish brown solution was formed. Dry palladium hydroxide (20% on carbon) (0.075 g, 15% wt/wt) was added to the solution under nitrogen atmosphere at 30° C. The solution was purged with hydrogen gas using a hydrogen balloon for 5 min at 30° C.

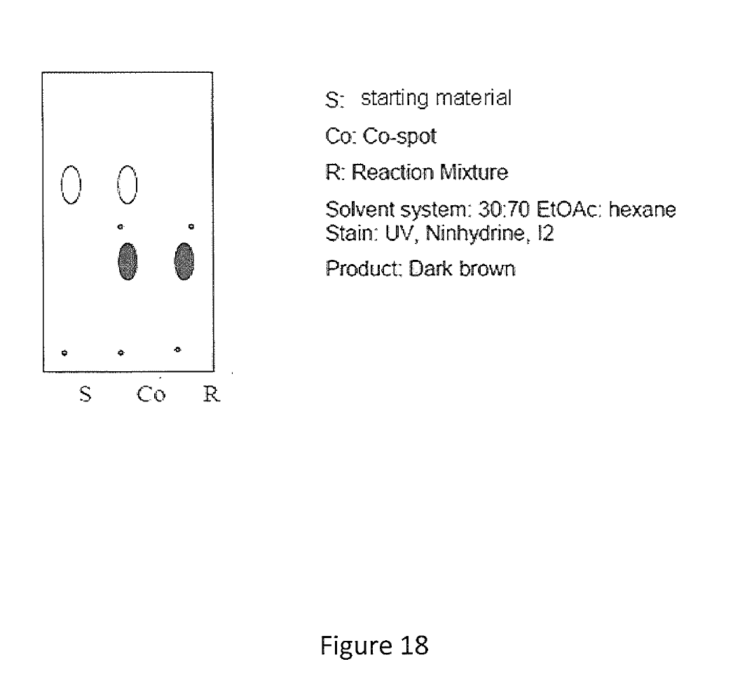

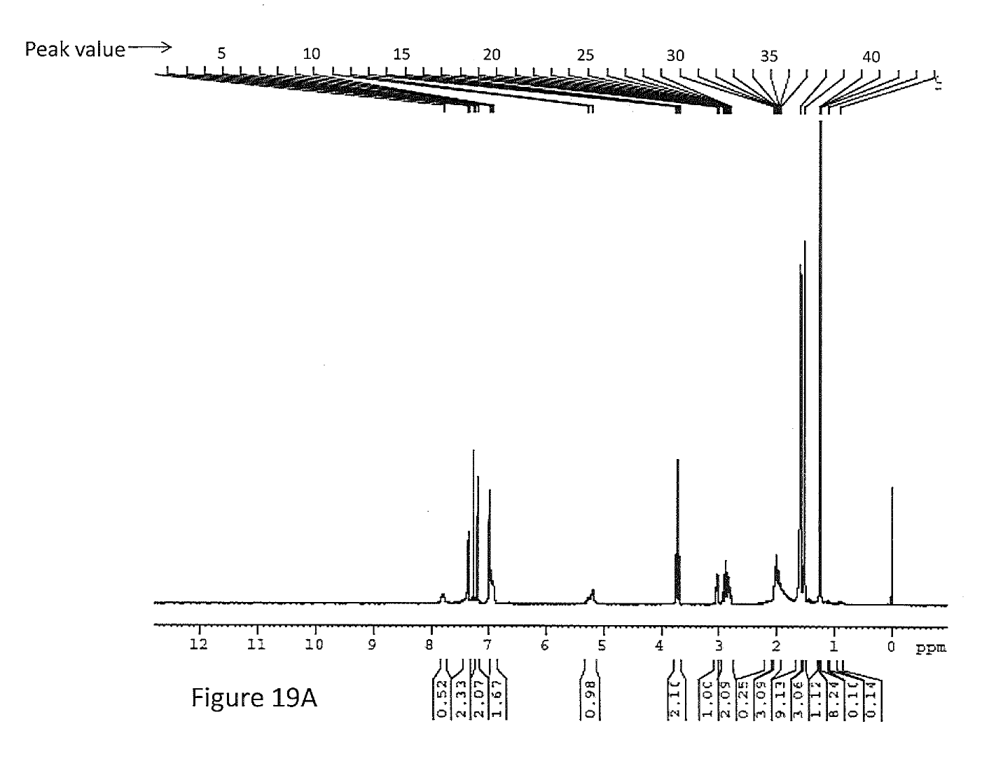

Hydrogenation was then started using a hydrogen balloon for 2 hours at 30° C. The reaction was monitored by TLC (FIG. 18) and HPLC. HPLC showed 98% conversion of starting material to product. In the TLC spotting method, ˜0.1 mL reaction mixture was filtered and the filtrate was used for TLC spotting.

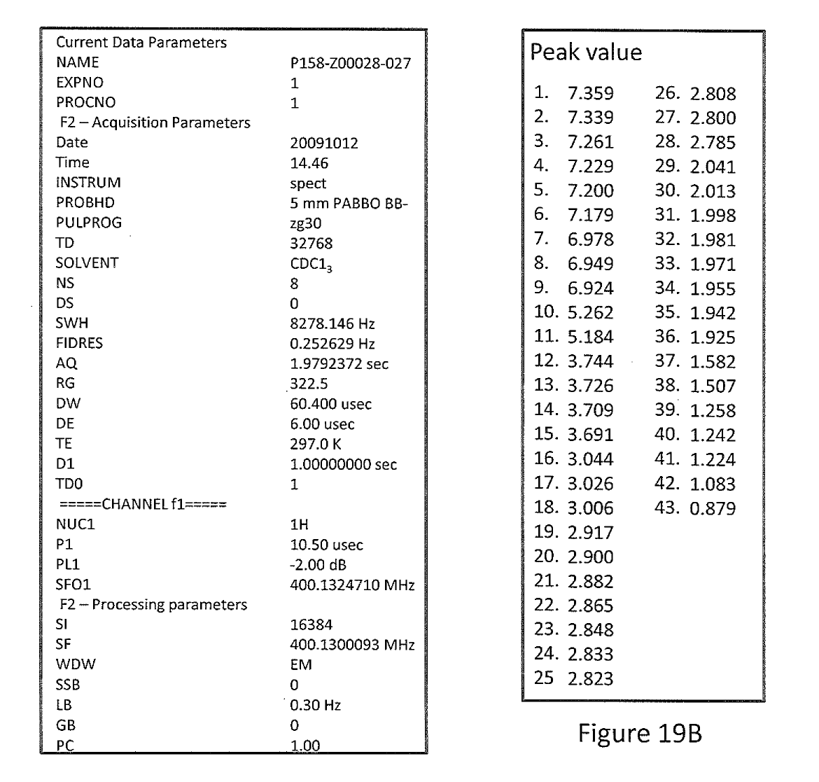

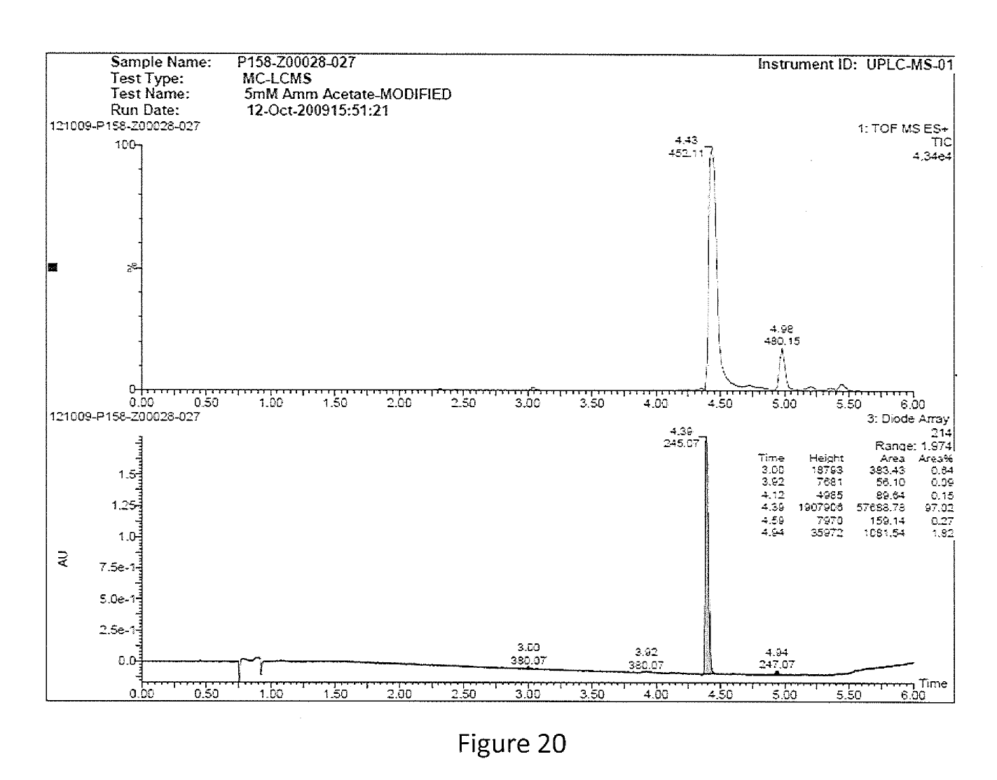

The reaction mixture was degassed by nitrogen gas using a nitrogen balloon, and filtered through a Celite bed. The Celite bed was washed with 30 mL ethanol, and the total filtrate liquid was concentrated at 40° C. under reduced pressure to afford 3.75 g yellowish white solid product. A ¹H NMR analysis of this compound (FIG. 19) confirmed the formation of desired 10. The yield was 90%, and purity was 97.02% by LC-MS (FIG. 20).

### Example 1

### (3aS)-3a-methyl-1,2,3,3a,8,8a-hexahydropyrrolo[2,3-b]indol-5-yl (4-isopropylphenyl)carbamate, 1

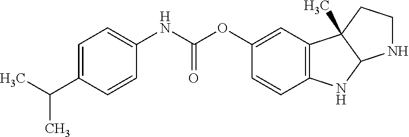 [SMILES: CC(C)c1ccc(NC(=O)Oc2ccc3c(c2)[C@]2(C)CCNC2N3)cc1]

Compound 10 (0.5 g, 0.0011 mol) was dissolved in dichloromethane under nitrogen atmosphere at 30° C. A yellowish brown color clear solution was formed. The reaction mixture was cooled to 0° C. Trifluoroacetic acid (1.01 g) was added dropwise over 10 minutes under nitrogen atmosphere at 0° C. A clear brown solution was formed. The reaction mixture was stirred for 5 hrs at 0° C. under nitrogen atmosphere.

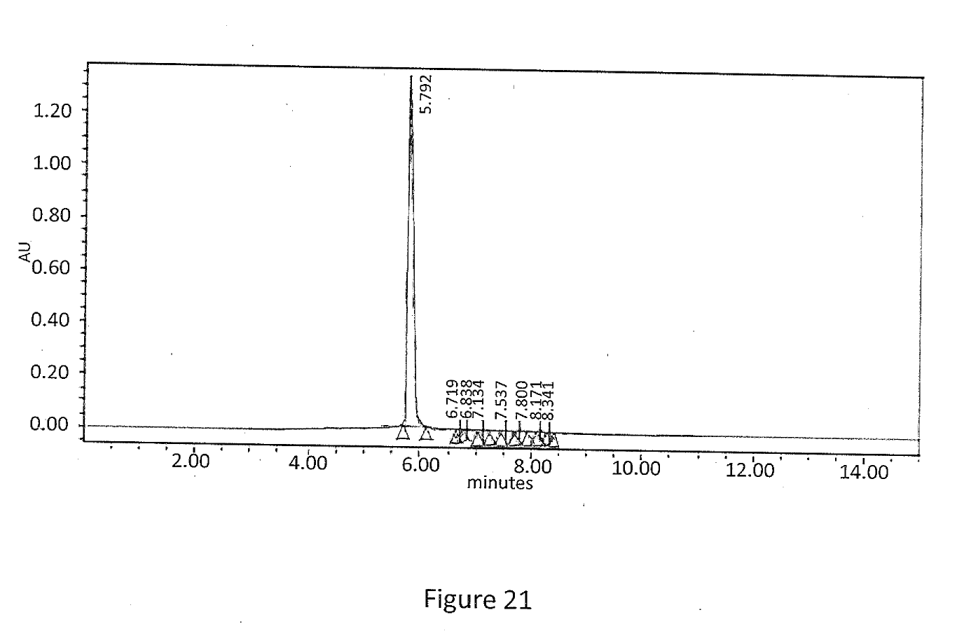

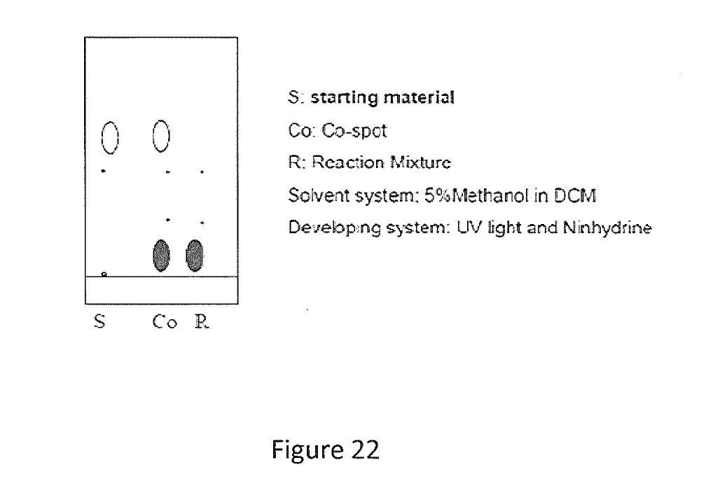

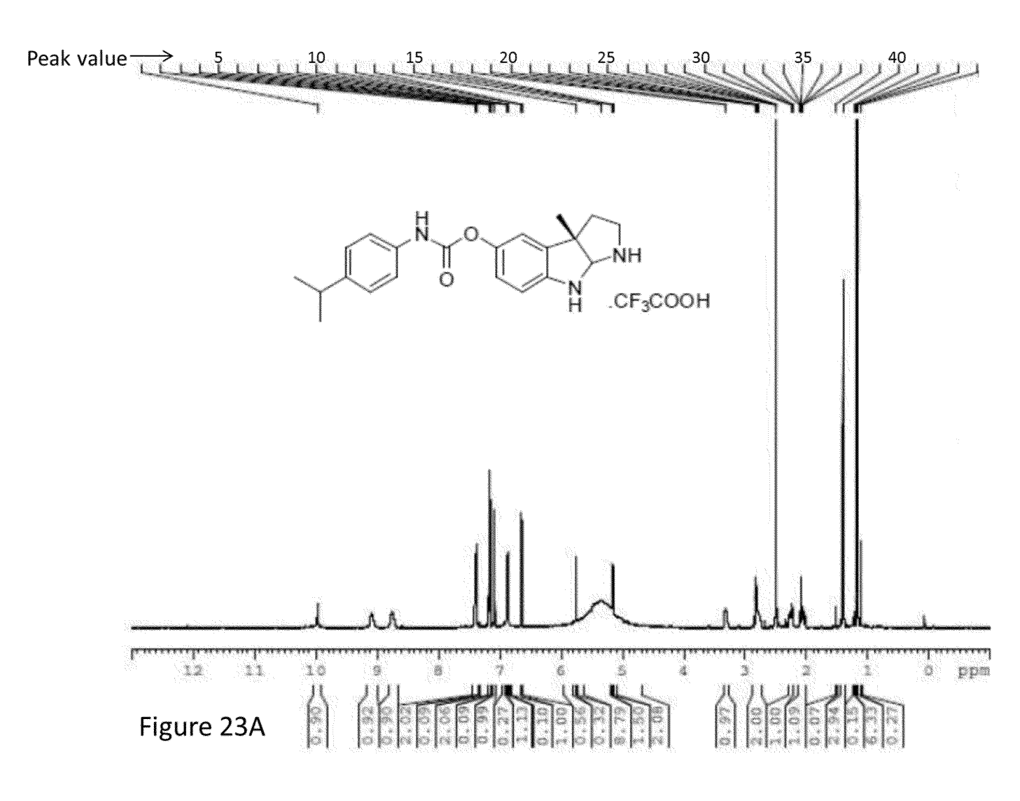

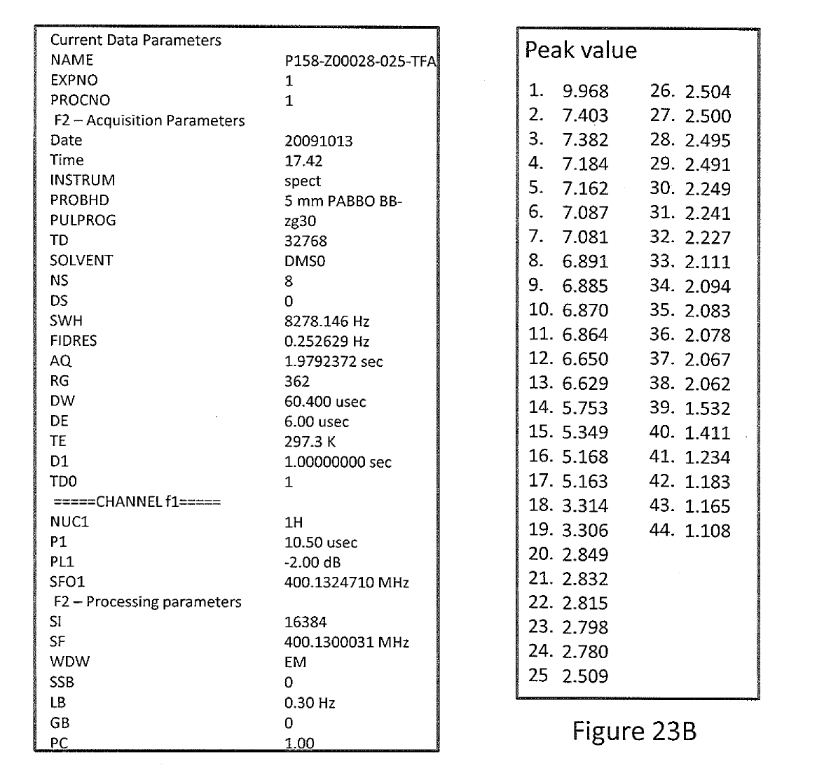

A representative HPLC trace of the reaction mixture is illustrated in FIG. 21. In order to prepare a TLC sample, 1 mL of the reaction mixture was treated with saturated sodium bicarbonate solution. After 1 mL of dichloromethane was added and the system was shaken thoroughly, the lower organic layer was used for TLC spotting (FIG. 21). A ¹H NMR spectrum of the crude trifluoroacetate salt is illustrated in FIG. 22.

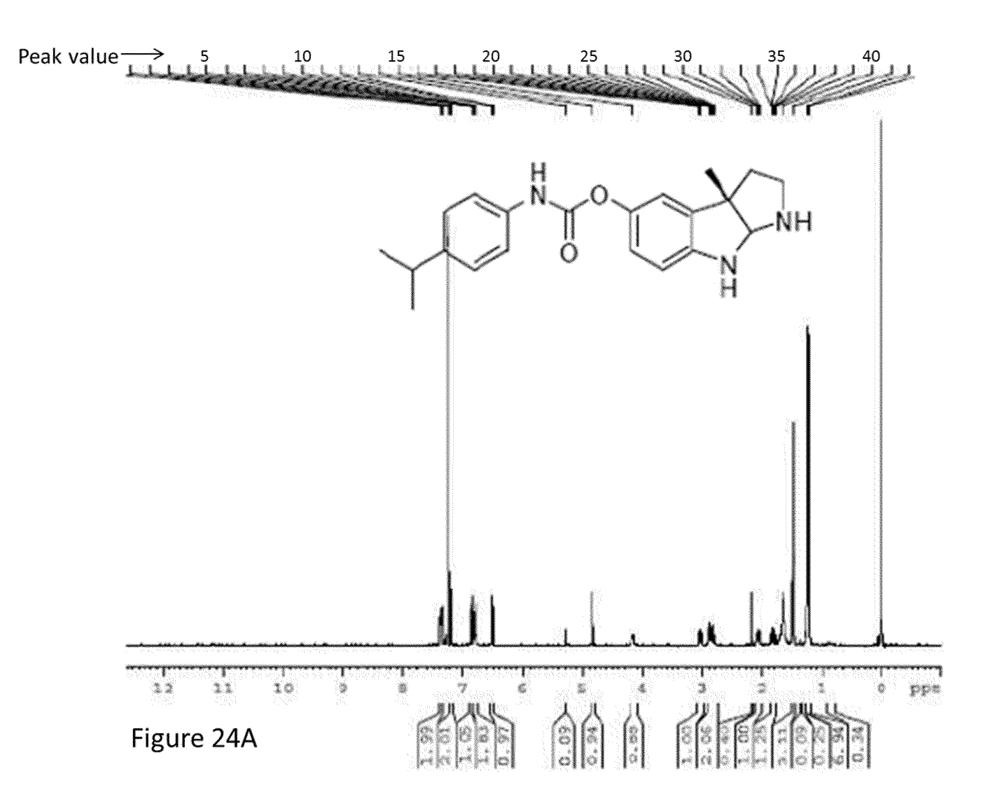

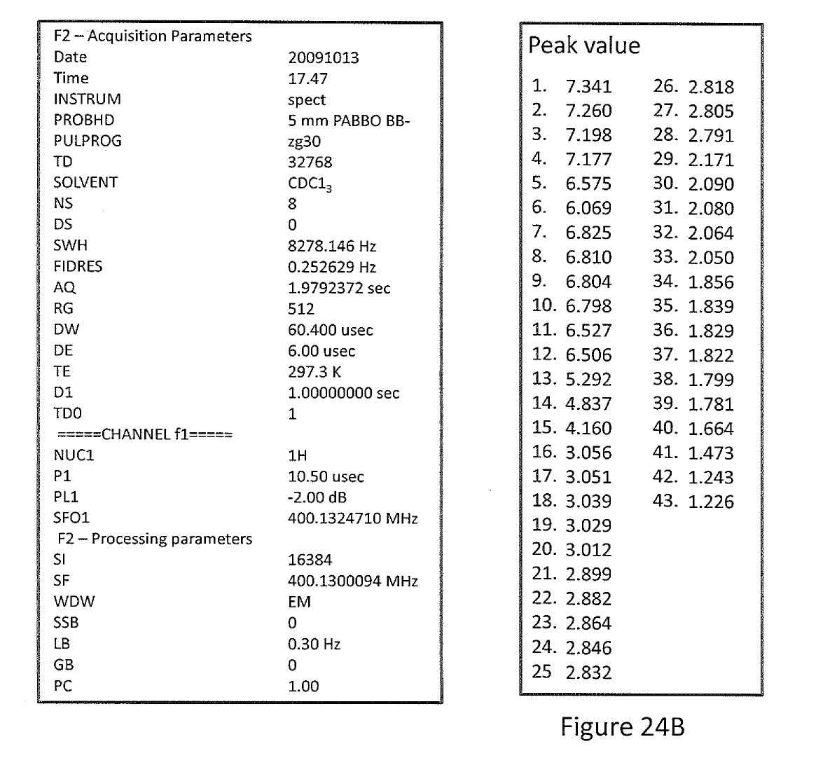

The reaction mixture was concentrated under vacuum at 44° C., yielding 450 mg crude brown oil. The crude brown oil was dissolved in dichloromethane (20.0 mL), and the reaction mixture was neutralized by adding 7.0 mL saturated sodium bicarbonate up to pH 7. The organic layer was then washed with 5.0 mL brine, dried over Na₂SO₄ and concentrated under vacuum at 44° C. 0.347 g white floppy compound product was obtained. A NMR spectrum of this material is illustrated in FIG. 24.

### Example 2

### (3aS)-3a-methyl-1,2,3,3a,8,8a-hexahydropyrrolo[2,3-b]indol-5-yl (4-isopropylphenyl) carbamate tartaric acid salt, 11

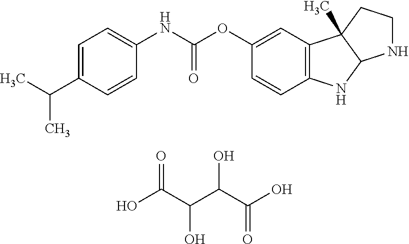 [SMILES: CC(C)c1ccc(NC(=O)Oc2ccc3c(c2)[C@]2(C)CCNC2N3)cc1 ; O=C(O)C(O)C(O)C(=O)O]

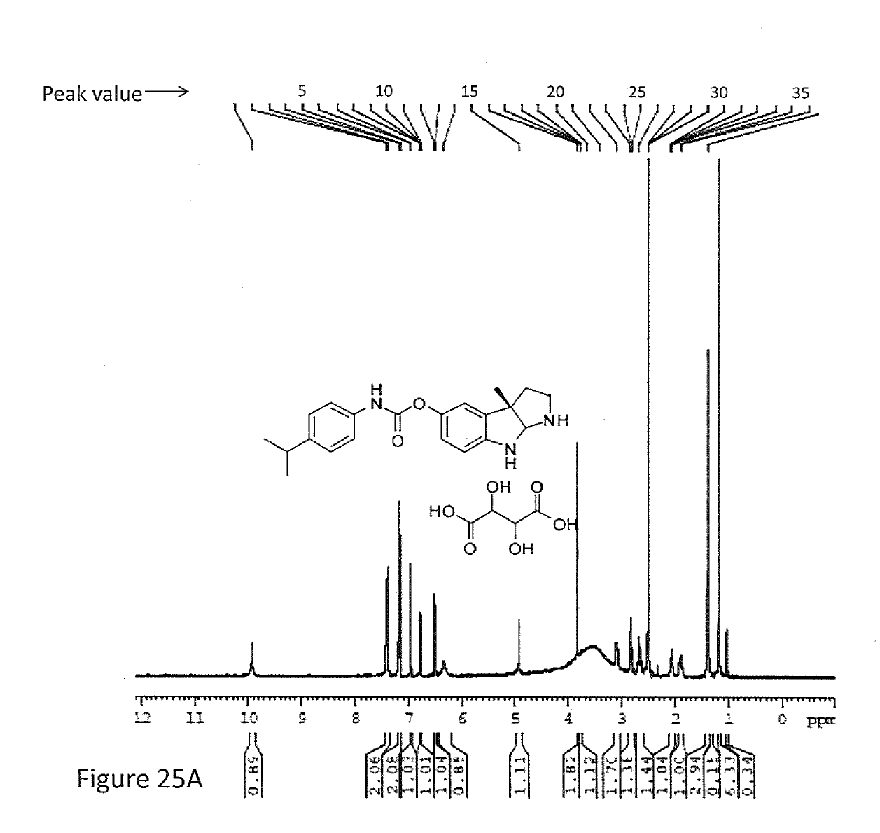

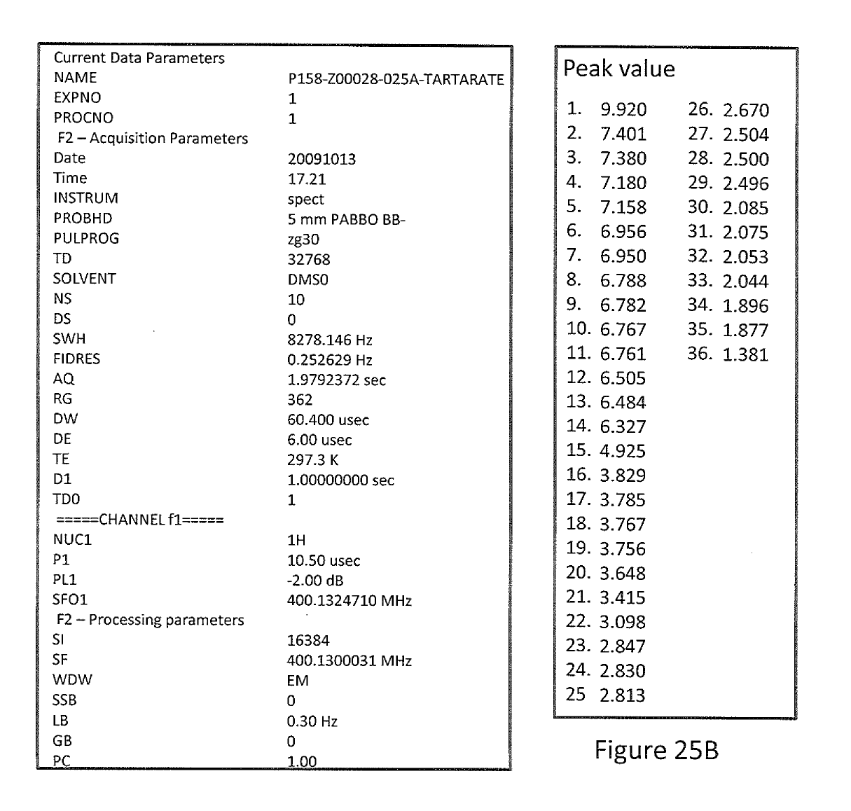

Pure compound 1 (0.347 g, 0.99 mmole) was dissolved in isopropanol (1.5 mL) at 30° C. (solution I). In a separate set up, tartaric acid (0.148 mg, 0.99 mmole) was dissolved in isopropanol (1.5 mL) at 55° C. (solution II), Solution I was heated up to 55° C., and solution II was added in a single lot to solution II. The reaction mixture was stirred for 30 min at 55° C. A white solid precipitated from the reaction mixture, and was filtered through a Buckner funnel. The product was dried under vacuum at 35° C. for 1 hour. The tartrate salt H (0.410 g) was obtained. A ¹H NMR spectrum of 11 in DMSO-d₆ is illustrated in FIG. 25.

The disclosures of each and every patent, patent application, and publication cited in the present patent application are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A composition comprising a compound of formula 10: 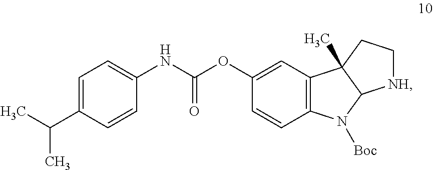 [SMILES: CC(C)c1ccc(NC(=O)Oc2ccc3c(c2)[C@]2(C)CCNC2N3C(=O)OC(C)(C)C)cc1]
or a salt thereof.

2. A method of preparing a salt comprising an acid and a compound of formula 1: 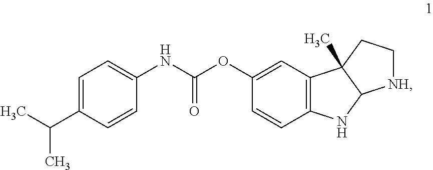 [SMILES: CC(C)c1ccc(NC(=O)Oc2ccc3c(c2)[C@]2(C)CCNC2N3)cc1]
said method comprising the steps of:
(i) dissolving one equivalent of said compound of formula 1 in a first volume of a first solvent, to generate a first solution;
(ii) dissolving a number of equivalents of said acid in a second volume of a second solvent, to generate a second solution;
(iii) contacting said second solution with said first solution under stirring, to generate a first system comprising a first solid;
(iv) stirring said first system at a first temperature for a first period of time;
(v) isolating said first solid from said first system by filtration;
(vi) washing said first solid with a third volume of a third solvent, to generate a second solid;
(vii) washing said second solid with a fourth volume of a fourth solvent, to generate a third solid;
(viii) washing said third solid with a fifth volume of a fifth solvent, to generate a fourth solid; and,
(ix) isolating and removing volatiles from said fourth solid, to generate said salt.

3. The method of claim 2, wherein said number of equivalents of said acid ranges from about 1 to about 3.

4. The method of claim 2, wherein in said salt the ratio of said acid to said compound of formula 1 ranges from about 1:1 to about 3:1.

5. The method of claim 2, wherein said acid is L-tartaric acid.

6. The method of claim 5, wherein in said salt the ratio of L-tartaric acid to said compound of formula 1 is about 1:1.

7. The method of claim 2, wherein said first solvent and said second solvent each comprise isopropanol.

8. The method of claim 7, wherein said first volume and said second volume are about 5 volumes each.

9. The method of claim 2, wherein said first temperature ranges from about 45 to about 75° C. and said first period of time is about one hour.

10. The method of claim 2, wherein said third solvent comprises isopropanol and said third volume is about 3 volumes.

11. The method of claim 2, wherein said fourth solvent comprises 10 volumes of DMSO and 22 volumes of water.

12. The method of claim 2, wherein said fifth solvent comprises acetonitrile and said fifth volume is about 11 volumes.

13. The method of claim 2, wherein said volatiles are removed by spray-drying or freeze-drying said fourth solid.

14. A method of preparing a compound of formula 1: 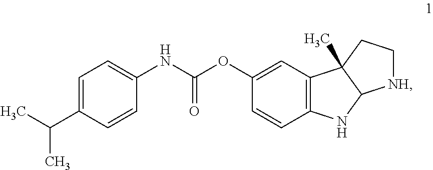 [SMILES: CC(C)c1ccc(NC(=O)Oc2ccc3c(c2)[C@]2(C)CCNC2N3)cc1]
or a salt thereof; comprising the step of hydrolyzing a compound of formula 10: 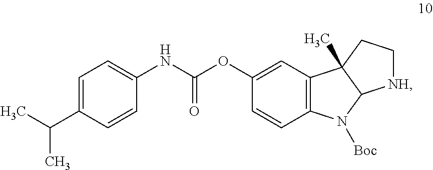 [SMILES: CC(C)c1ccc(NC(=O)Oc2ccc3c(c2)[C@]2(C)CCNC2N3C(=O)OC(C)(C)C)cc1]
or a salt thereof.

15. The method of claim 14, wherein said compound of formula 10 is hydrolyzed with a solution of trifluoroacetic acid in dichloromethane.

16. The method of claim 14, wherein said compound of formula 10 or a salt thereof is prepared from a compound of formula 9 or a salt thereof: 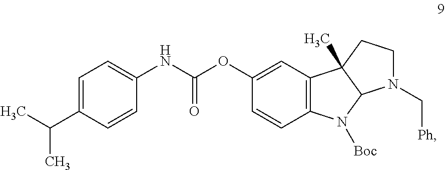 [SMILES: CC(C)c1ccc(NC(=O)Oc2ccc3c(c2)[C@]2(C)CCN(Cc4ccccc4)C2N3C(=O)OC(C)(C)C)cc1]
by hydrogenating said compound of formula 9.

17. The method of claim 16, wherein said compound of formula 9 or a salt thereof is prepared from a compound of formula 8 or a salt thereof: 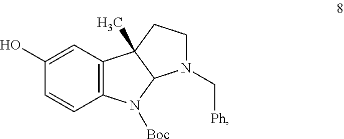 [SMILES: CC(C)(C)OC(=O)N1c2ccc(O)cc2[C@]2(C)CCN(Cc3ccccc3)C12]
by reacting said compound of formula 8 with:
(i) isopropyl isocyanate in the presence of an organic base; or,
(ii) a reagent selected from the group consisting of phosgene, diphosphene, triphosgene, carbonyldiimidazole and para-nitrophenyl chloroformate, to form an intermediate, and further reacting said intermediate with para-isopropylaniline.

18. The method of claim 17, wherein said compound of formula 8 or a salt thereof is prepared from a compound of formula 7 or a salt thereof: 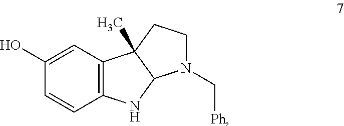 [SMILES: C[C@@]12CCN(Cc3ccccc3)C1Nc1ccc(O)cc12]
by reacting said compound of formula 7 with a BOC-protecting reagent.

19. The method of claim 18, wherein said compound of formula 7 or a salt thereof is prepared from a compound of formula 6 or a salt thereof: 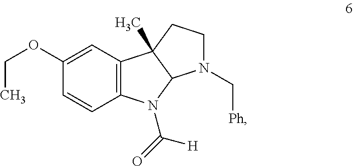 [SMILES: CCOc1ccc2c(c1)[C@]1(C)CCN(Cc3ccccc3)C1N2C=O]
by reacting said compound of formula 6 with a reagent selected from the group consisting of boron tribromide, trimethylsilyl iodide, trimethylsilyl chloride, trifluoroboron etherate, tetrachlorosilane, aluminum tribromide, aluminum trichloride, ferric trichloride, and bromodimethylborane.

20. The method of claim 19, wherein said compound of formula 6 or a salt thereof is prepared from a compound of formula 5 or a salt thereof: 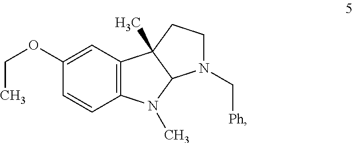 [SMILES: CCOc1ccc2c(c1)[C@]1(C)CCN(Cc3ccccc3)C1N2C]
by reacting said compound of formula 5 with an oxidizer.

21. The method of claim 20, wherein said compound of formula 5 or a salt thereof is prepared from a compound of formula 4 or a salt thereof: 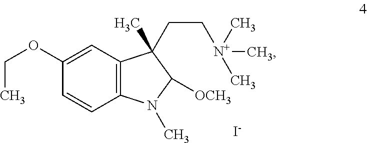 [SMILES: CCOc1ccc2c(c1)[C@](C)(CC[N+](C)(C)C)C(OC)N2C ; [I-]]
by reacting said compound of formula 4 with benzylamine.

22. The method of claim 21, wherein said compound of formula 4 or a salt thereof is prepared from a compound of formula 3 or a salt thereof: 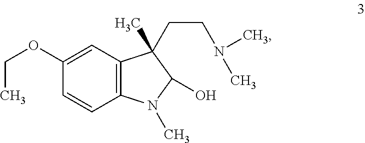 [SMILES: CCOc1ccc2c(c1)[C@](C)(CCN(C)C)C(O)N2C]
by reacting said compound of formula 3 with methyl iodide.

23. The method of claim 22, wherein said compound of formula 3 or a salt thereof is prepared from a compound of formula 2 or a salt thereof: 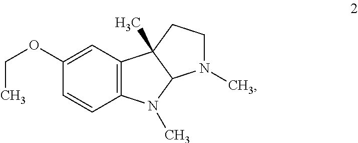 [SMILES: CCOc1ccc2c(c1)[C@]1(C)CCN(C)C1N2C]
comprising the steps of:
(i) reacting said compound of formula 2 with a methylating agent, to generate a methylated derivative of said compound of formula 2, and
(ii) further reacting said methylated derivative with an aqueous solution comprising an inorganic base,
whereby generating said compound of formula 3.
